(19) United States
(12) Patent Application Publication
(10) Pub. No.: US 2019/0323053 A1
(43) Pub. Date: Oct. 24, 2019

(54) INCREASING ACTIVITY OF 2? FUCOSYLLACTOSE TRANSPORTERS ENDOGENOUS TO MICROBIAL CELLS

(71) Applicant: DUPONT NUTRITION BIOSCIENCES APS, Copenhagen (DK)

(72) Inventors: Lisa A LAFFEND, CALYMONT, DE (US); Mark J. Nelson, Newark, DE (US); Lori Ann Maggio-Hall, Wilmingon, DE (US)

(21) Appl. No.: 15/959,573

(22) Filed: Apr. 23, 2018

(51) Int. Cl. C12P 21/00; C12N 15/67; A61K 31/702; C12N 15/70

(52) U.S. Cl. CPC C12P 21/005; C12N 15/70; A61K 31/702; C12N 15/67

(57) ABSTRACT

A fermentation broth which includes a microbial cell which has been subjected to a condition under which the activity an endogenous transporter for 2′ fucosyllactose is increased. Also provided are methods for increasing export of 2′ fucosyllactose from a microbial cell, methods for identifying an endogenous yeast transporter of 2′ fucosyllactose, and microbial cells genetically engineered to increase the activity of an endogenous transporter of 2′ fucosyllactose.

### INCORPORATION BY REFERENCE OF THE SEQUENCE LISTING

The sequence listing provided in the file named “NB06492USNP_SequenceListing_ST25” with a size of 50,104 bytes which was created on Apr. 19, 2018 and which is filed herewith, is incorporated by reference herein in its entirety.

### FIELD

The disclosure relates to the modification of growth conditions and genetic engineering of microbial cells to increase the activity of endogenous transporters of 2′ fucosyllactose.

### BACKGROUND

2′ fucosyllactose (2′FL) is a human milk oligosaccharide (HMO) shown to be beneficial to infant health. E. coli has been genetically engineered to produce 2′FL by introducing a biosynthetic pathway to GDP-L-fucose, which is then combined with lactose by catalytic action of an α-1,2-fucosyltransferase to generate 2′FL (Lee et al. (2012) Microb. Cell Factories 11, 48-57; Baumgartner et al. (2013) 12, 40-53; US Patent Application 20140024820). U.S. Pat. No. 8,652,808 discloses a bacterial cell engineered to synthesize 2′FL and a sugar efflux transporter to excrete it to the medium. In addition, others have established a metabolic route to GDP-fucose in Corynebacterium glutamicum that could enable production of 2′FL or other fucosylated HMOs (Chin et al (2013) Bioprocess Biosyst. Eng 36, 749-756).

A metabolic route to GDP-fucose has been established in Saccharomyces cerevisiae (Matila et al. (2000) Glycobiology 10, 1041-1047)), and the synthesis of 2′FL in Kluyveromcyes lactis has been reported as a method to demonstrate successful synthesis of GDP-fucose (US Patent Application 2010/0120701). However, Applicants are unaware of a reported method for increasing the endogenous activity of a 2′FL transporter in microbial cells.

### SUMMARY

In one aspect, the disclosure provides a fermentation broth which includes a microbial cell having increased export of 2′ fucosyllactose, the microbial cell having been subjected to a condition under which the activity of an endogenous transporter is increased relative to the activity of the endogenous transporter in the microbial cell in the absence of subjecting the microbial cell to the condition.

In another aspect, the disclosure provides a method for increasing the export of 2′ fucosyllactose from a microbial cell. The method includes the steps of a) obtaining a 2′FL-containing microbial cell and b) subjecting the microbial cell to a condition under which the activity of an endogenous transporter is increased relative to the activity of the endogenous transporter in the microbial cell in the absence of subjecting the microbial cell to the condition.

In a further aspect, the disclosure provides a method for identifying an endogenous yeast transporter for exporting 2′ fucosyllactose from a yeast cell. The method includes the steps of a) obtaining a 2′FL-containing yeast cell and b) subjecting the yeast cell to a condition under which the export of 2′FL is increased relative to the export of 2′FL in the yeast cell in the absence of subjecting the yeast cell to the condition. The method further includes the step of identifying an endogenous yeast transporter with increased activity in the yeast cell as an endogenous yeast transporter for exporting 2′ fucosyllactose.

In yet a further aspect, the disclosure provides a genetically engineered microbial cell. The genetically engineered microbial cell includes a genetic modification which increases the activity of one or more endogenous transporters whereby export of 2′ fucosyllactose from the microbial cell is increased.

### BRIEF DESCRIPTION OF THE DRAWINGS AND SEQUENCE DESCRIPTIONS

FIG. 1 shows a diagram of a biosynthetic pathway for production of 2′FL.

FIG. 2 (A-C) shows a comparison of 2′ fucosyllactose export from yeast cells in glucose excess versus glucose limited growth conditions.

FIG. 3 (A-C) shows a comparison of 2′ fucosyllactose export from yeast cells grown in media supplemented with glucose or ethanol.

FIG. 4 (A-C) shows a comparison of 2′ fucosyllactose export from yeast cells grown in synthetic complete versus minimal media.

The disclosure can be more fully understood from the following detailed description and the accompanying sequence descriptions which form a part of this application.

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. § 1.822.

SEQ ID NO:1 is the nucleotide sequence of the coding region for lactose permease from Kluyveromyces lactis.

SEQ ID NOs:2, 3, 5, 6, 8, 9, 11-15, 19-22, 28-31, 38-41, and 43-46 are PCR and/or sequencing primers.

SEQ ID NO:4 is the nucleotide sequence of the PMA1 promoter.

SEQ ID NO:7 is the nucleotide sequence of theTPS1 terminator.

SEQ ID NO:10 is the nucleotide sequence of plasmid pUC19-URA3-YPRCΔ15.

SEQ ID NO:17 is the nucleotide sequence of a Kluyveromyces lactis beta-galactosidase 5′ fragment.

SEQ ID NO:18 is the nucleotide sequence of a Kluyveromyces lactis beta-galactosidase 3′ fragment.

SEQ ID NO:23 is the nucleotide sequence of plasmid pHR81-ILV5p-R8B2y2.

SEQ ID NO:24 is the nucleotide sequence of the ILV5 promoter.

SEQ ID NO:25 is the nucleotide sequence of the ILV5 terminator.

SEQ ID NO:26 is the nucleotide sequence of the coding region for GDP-mannose dehydratase from E. coli.

SEQ ID NO:27 is the nucleotide sequence of the coding region for GDP-4-keto-6-deoxymannose epimerase reductase from E. coli.

SEQ ID NO:32 is the nucleotide sequence of the PDC1 promoter.

SEQ ID NO:33 is the nucleotide sequence of the ADH1 terminator.

SEQ ID NO:34 is the nucleotide sequence of the hybrid promoter (PGK1(UAS)-FBA1).

SEQ ID NO:35 is the nucleotide sequence of the TDH3 terminator.

SEQ ID NO: 36 is the nucleotide sequence of the coding region for GDP-mannose dehydratase from A. thaliana.

SEQ ID NO: 37 is the nucleotide sequence of the coding region for GDP-4-keto-6-deoxymannose epimerase reductase from A. thaliana.

SEQ ID NO:42 is the nucleotide sequence of the coding region for FutC from Helicobacter pylori with BsaI sites on the ends.

SEQ ID NO:16 is the nucleotide sequence of the coding region for beta-galactosidase from Kluyveromyces lactis.

### DETAILED DESCRIPTION

The following definitions may be used for the interpretation of the claims and specification:

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. The compositions and methods disclosed herein may comprise, consist, or consist essentially of any element disclosed herein. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

“Gene” refers to a nucleic acid fragment that expresses a specific protein or functional RNA molecule, which may optionally include regulatory sequences preceding (5′ non-coding sequences) and following (3′ non-coding sequences) the coding sequence. “Native gene” or “wild type gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature.

The term “endogenous gene” refers to a native gene of an organism. A “foreign” gene refers to a gene not normally found in the host organism, but that is introduced into the host organism by gene transfer. Foreign genes can comprise native genes inserted into a non-native organism, or chimeric genes.

“Promoter” or “Initiation control regions” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in a cell type at most times are commonly referred to as “constitutive promoters”.

The term “expression”, as used herein, refers to the transcription and stable accumulation of coding (mRNA) or functional RNA derived from a gene. Expression may also refer to translation of mRNA into a polypeptide. “Overexpression” refers to the production of a gene product in transgenic organisms that exceeds levels of production in normal or non-transformed organisms.

The term “transformation” as used herein, refers to the transfer of a nucleic acid fragment into a host organism, resulting in genetically stable inheritance. The transferred nucleic acid may be in the form of a plasmid maintained in the host cell, or some transferred nucleic acid may be integrated into the genome of the host cell. Host organisms containing the transferred nucleic acid fragments are referred to as “transgenic” or “recombinant” or “transformed” organisms or “transformants”.

The terms “plasmid” and “vector” as used herein, refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “selectable marker” means an identifying factor, usually an antibiotic or chemical resistance gene, that is able to be selected for based upon the marker gene's effect, i.e., resistance to an antibiotic, wherein the effect is used to track the inheritance of a nucleic acid of interest and/or to identify a cell or organism that has inherited the nucleic acid of interest.

As used herein the term “codon degeneracy” refers to the nature of the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it may be desirable to design the gene such that its frequency of codon usage approaches the frequency of preferred codon usage of the host cell.

The term “codon-optimized” as it refers to genes or coding regions of nucleic acid molecules for transformation of various hosts, refers to the alteration of codons in the gene or coding regions of the nucleic acid molecules to improve the production of the polypeptide encoded by the DNA without altering the sequence of the polypeptide.

The term “heterologous” means not naturally found in the cellular location of interest. For example, a heterologous gene refers to a gene that is not naturally found in the host organism, but that is introduced into the host organism by gene transfer. For example, a heterologous nucleic acid molecule that is present in a chimeric gene is a nucleic acid molecule that is not naturally found associated with the other segments of the chimeric gene, such as the nucleic acid molecules having the coding region and promoter segments not naturally being associated with each other.

As used herein, an “isolated nucleic acid molecule” is a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Press, Cold Spring Harbor, N.Y. (1984); and by Ausubel, F. M. et. al., Short Protocols in Molecular Biology, 5^{th} Ed. Current Protocols, John Wiley and Sons, Inc., N.Y., 2002. Additional methods used here are in Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.).

In one aspect, the disclosure provides a fermentation broth which includes a microbial cell having increased export of 2′ fucosyllactose, the microbial cell having been subjected to a condition under which the activity of an endogenous transporter is increased relative to the activity of the endogenous transporter in the microbial cell in the absence of subjecting the microbial cell to the condition.

The fermentation broth can be any fermentation broth in which microbial cells can be fermented such as those known in the art. A fermentation broth will typically contain suitable carbon substrates, most typically glucose, but may contain other carbon sources, such as the non-fermentable carbon sources disclosed below. Carbon substrates may be provided by glucose preparations or by glucose and other sugars prepared from starch biomass or lignocellulosic biomass. A starch biomass, such as ground corn grain, is typically treated using alpha amylase and glucoamylase enzymes to prepare a hydrolyzed mash that can be used as fermentation medium. A lignocellulosic biomass is typically pretreated with mechanical energy and chemicals, then hydrolyzed using multiple glycosidases including cellulases and other enzymes, such as disclosed in WO 2011/038019, to produce a lignocellulosic biomass hydrolysate containing glucose, xylose, and arabinose that can be used as fermentation medium, for example as disclosed in U.S. Pat. No. 7,932,063. In certain embodiments, the fermentation broth includes one or more non-fermentable carbon sources such as ethanol, glycerol, and acetate. The fermentation broth may include these non-fermentable carbon sources in addition to or in place of fermentable carbon sources like glucose.

Specific attributes of the fermentation broth and fermentation conditions will be determined by the type of microbial cell used. One of skill in the art will be familiar with conditions such as pH, oxygenation, and temperature used for various bacterial and fungal cells.

In certain embodiments, the condition for increasing activity of an endogenous transporter in the microbial cell is a growth condition. In certain embodiments, the growth condition is a condition involving the components of the medium in which the microbial cell is grown, such as components of the fermentation broth. In specific embodiments, increased activity of an endogenous transporter is obtained by growing the microbial cell in a medium which has one or more of the following characteristics: the medium includes amino acids, the medium is a glucose limited medium, and the medium includes ethanol. Media with these characteristics can be made by methods known in the art, such as the exemplary media described in Example 3. In a specific embodiment, the medium includes amino acids and at least one of folic acid, riboflavin, micronutrients, and adenine. In a particularly specific embodiment, the medium is the synthetic complete medium described in Example 3. Where the endogenous transporter is a transporter capable of exporting 2′ fucosyllactose from the microbial cell, increased activity of the transporter would, in turn, result in increased export of 2′ fucosyllactose present in the microbial cell. Therefore, by subjecting the microbial cell to the condition, a microbial cell, as present in the fermentation broth, with increased export of 2′ fucosyllactose is obtained.

In that regard, in another aspect, the disclosure provides a method of increasing export of 2′ fucosyllactose from a microbial cell. The method includes the steps of a) obtaining a 2′FL-containing microbial cell and b) subjecting the microbial cell to a condition under which the activity of an endogenous transporter is increased relative to the activity of the endogenous transporter in the microbial cell in the absence of subjecting the microbial cell to the condition.

The microbial cell can be any microbial cell from which 2′ fucosyllactose can be exported. In certain embodiments, the microbial cell is a bacterial cell or a fungal cell. In particular embodiments, the bacterial cell is of the genus such Escherichia, Bacillus, Methylomonas, Pseudomonas, Lactobacillus, or Corynebacterium. In various embodiments the microbial cell is an Escherichia coli or Bacillus subtilis cell. In certain embodiments, the microbial cell is a yeast cell. In certain embodiments, the yeast cell is of the genus Saccharomyces, Yarrowia, Kluyveromyces, Candida, Hansenula, Pichia, Schizosaccharomyces, Zygosaccharomyces, Debaryomyces, Brettanomyces, Pachysolen, Issatchenkia, Trichosporon, or Yamadazyma. In various embodiments the yeast cell is from Saccharomyces cerevisiae, Yarrowia lipolytica or Kluyveromyces lactis.

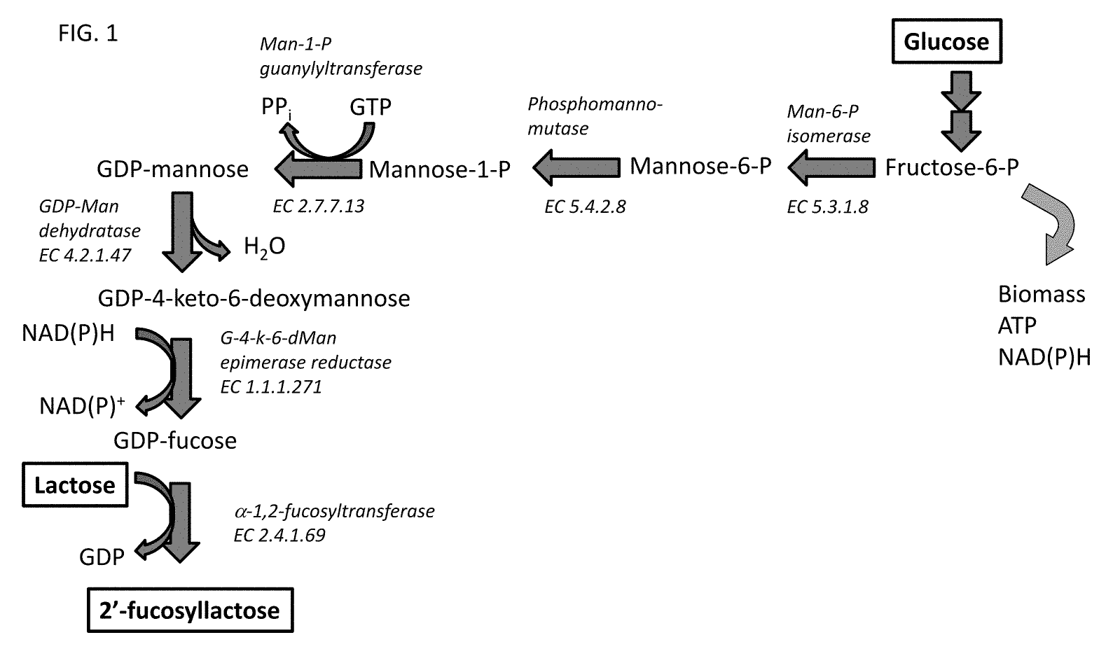

In certain embodiments, the microbial cell is a cell that is genetically engineered to produce 2′FL, i.e., is a 2′FL-producing cell. Methods for genetically engineering E. coli cells to produce 2′FL have been previously described (Lee et al. (2012) Microb. Cell Factories 11, 48-57; Baumgartner et al. (2013) 12, 40-53; US Patent Application 20140024820). Methods for genetically engineering yeast cells, such as yeasts of the genera Saccharomyces, Yarrrowia, Kluyveromyces, Pichia, and Hansenula, to produce 2′FL are disclosed herein. In certain embodiments, yeast cells capable of producing 2′FL are constructed as described in Example 1. Specifically, the Example discloses a method in which 2′FL producing yeast cells are made by expressing heterologous coding regions for GDP-mannose-4,6-dehydratase (GMD; EC 4.2.1.47), GDP-4-keto-6-D-deoxymannose epimerase-reductase (GDP-L-fucose synthase; GMER; EC 1.1.1.271), and 2-α-L-fucosyltransferase (2FT; EC 2.4.1.69) in a yeast host that has a native pathway to GDP-mannose, and then supplying a source of lactose for the 2FT reaction. The native yeast pathway to GDP-mannose optionally may be enhanced by increasing expression of the endogenous pathway enzymes using methods described below. This pathway is shown in FIG. 1.

As shown in FIG. 1, one method of producing 2′FL uses an α-1,2-fucosyltransferase to catalyze the combination of lactose and GDP-fucose. In certain embodiments, it is expected that increasing the ability of a microbial cell to import lactose will result in increased 2′FL within the microbial cell resulting in greater 2′FL available for export. In certain embodiments, in addition to being genetically engineered to express the pathway enzymes for 2′FL production, the microbial cell is genetically engineered to include a nucleic acid sequence which codes for a lactose transporter. The lactose transporter can be any lactose transporter known in the art that can be expressed in the microbial cell such as the lactose transporter encoded by SEQ ID NO: 1.

The fermentation broth may contain additional substrates that contribute to production of the desired product. For example, where the microbial cell is a 2′FL-producing cell, the broth may contain lactose which can facilitate the production of fucosyllactose (see FIG. 1). Typically, these substrates would be provided by a batch feeding process as is known in the art.

2′FL-producing yeast cells are constructed according to methods well-known to one skilled in the art. Expression of heterologous coding regions in a host cell is known to one of skill in the art. The coding region for the desired polypeptide is readily obtained from the genome of the cell in which it is natively expressed, as well known to one skilled in the art. In addition, coding regions may be synthesized using codon optimization for the target host cell. Typically the nucleotide sequence encoding the amino acid sequence of the enzyme with desired activity is operably linked in a chimeric gene (or expression cassette) to a promoter that is active in the target host cell. Typically a transcription terminator is linked at the 3′ end of the coding region. For example, for expression in a yeast cell a number of yeast promoters can be used in constructing chimeric genes encoding a desired enzyme, including, but not limited to constitutive promoters FBA1, GPD1, ADH1, GPM, TPI1, TDH3, PGK1, Ilv5, and the inducible promoters GAL1, GAL10, and CUP1. Suitable transcription terminators include, but are not limited to FBAt, GPDt, GPMt, ERG10t, GAL1t, CYC1t, ADH1t, TALI t, TKL1t, ILV5t, and ADHt.

A chimeric gene for host cell expression is typically constructed in or transferred to a vector for further manipulations. The vector used is determined by the target host cell, and the transformation and/or integration methods to be used. Vectors for a target host cell are well known in the art. For example, for yeast expression chimeric genes may be cloned into E. coli-yeast shuttle vectors, and transformed into yeast cells. These vectors allow propagation in both E. coli and yeast cells. Typically the vector contains a selectable marker and sequences allowing autonomous replication or chromosomal integration in the desired host. Plasmids for DNA integration may include transposons, regions of nucleic acid sequence homologous to the target genome, or other sequences supporting integration. It is well known how to choose an appropriate vector for the desired target host and the desired function. In addition, a selectable marker used to obtain transformed cells may be bounded by site-specific recombination sites, so that after expression of the corresponding site-specific recombinase, the resistance gene is excised from the genome. Multiple copies of gene may be introduced on a plasmid or integrated into the cell genome.

There are many tests to determine if a microbial cell has increased export of 2′FL. For example, the export of 2′FL from a strain that synthesizes it (i.e., a 2′FL-producing cell) can be measured by detecting it in the broth of fermentations under conditions in which the 2′FL is being synthesized inside the cell. The 2′FL can be detected directly by means of chromatography of clarified broth samples removed from the fermentation, followed by detection by, for example, evaporative light scattering detection. The 2′FL can also be detected in clarified broth samples indirectly by means of a coupled enzyme assay, first catalyzing hydrolysis of the 2′FL with an α-1,2-L-fucosidase enzyme (EC 3.2.1.63) and then catalyzing oxidization of the resulting fucose to fuconate with an NAD⁺-dependent L-fucose dehydrogenase enzyme (EC 1.1.1.122), and detecting the product NADH spectrophotometrically. 2′FL export may be measured indirectly based on a change in pH if the heterologous nucleic acid sequence encodes a protein which moves H+ during 2′FL export. The use of antibodies to detect products of fermentation reactions by ELISA-type assays are well known in the art, as is the analogous use of RNA-aptamers specific for the desired product. Higher throughput screens could be available by screening the growth rates of strains engineered to make 2′FL with different heterologous nucleic acid sequences, as it is to be expected that buildup of an osmolyte such as 2′FL will cause stress that will inhibit cell growth, or that buildup of pathway intermediates will be otherwise deleterious to cell growth.

The above-described methods for detecting 2′ fucosyllactose can be used to identify microbial cells having increased export of 2′ fucosyllactose. Microbial cells having increased export of 2′ fucosyllactose can be identified by determining an amount of 2′ fucosyllactose present in a fermentation broth of a first microbial cell subjected to a condition, such as the growth conditions described above for increasing 2′ fucosyllactose export. The amount of 2′ fucosyllactose in the fermentation broth can then be compared to an amount of 2′ fucosyllactose in the fermentation broth of a second microbial cell that was not subjected to the growth condition. In certain embodiments, the first and second microbial cells are the same cell, e.g., where the amount of 2′ fucosyllactose is measured at different time points, e.g., prior to and after subjecting the microbial cell to the condition. In certain embodiments, the first and second microbial cells are different cells. Where the microbial cells are different cells, the microbial cells will be microbial cells of the same genus and species, and, in particular embodiments, will be derived from the same parental strain. The comparison of the amount of 2′ fucosyllactose in the fermentation broths for the first and second cells can be carried out on an absolute basis, e.g., based on the absolute concentration of 2′ fucosyllactose in the fermentation broths, or on a relative basis. A relative analysis can be carried out by measuring an amount of 2′ fucosyllactose in the fermentation broths of the first and second microbial cells relative to the amounts of 2′ fucosyllactose in the first and second cells. When using a relative analysis, a microbial cell having increased 2′ fucosyllactose export can be identified as a cell with a greater relative concentration of 2′ fucosyllactose in the fermentation broth to the concentration of 2′ fucosyllactose in the cell. This can be done, for example, by calculating a percentage or ratio of the amount of 2′ fucosyllactose in the fermentation broth for each of the first and second cells relative to the corresponding cellular concentrations and identifying the cell associated with a fermentation broth having a greater percentage or ratio of 2′ fucosyllactose. In certain embodiments, the cell having increased export of 2′FL has increased export of 2′FL relative to another cell that also exports 2′FL. In certain embodiments, the cell having increased export of 2′FL has increased export of 2′FL relative to another cell that does not export 2′FL. Microbial cells having increased export of 2′ fucosyllactose can, in turn, be used to identify endogenous transporters of 2′ fucosyllactose.

In that regard, a further aspect of the disclosure relates to a method of identifying an endogenous yeast transporter for exporting 2′ fucosyllactose from a yeast cell. The method includes the steps of a) obtaining a 2′FL-containing yeast cell and b) subjecting the yeast cell to a condition under which the export of 2′FL is increased relative to the export of 2′FL in the yeast cell in the absence of subjecting the yeast cell to the condition. The method further includes the step of identifying an endogenous yeast transporter with increased activity in the yeast cell as an endogenous yeast transporter for exporting 2′ fucosyllactose.

The yeast cell can be any yeast cell that contains 2′FL, including a yeast cell from any yeast disclosed herein. The condition to which the yeast cell is subjected can be any condition which results in an increase in 2′FL export, including any growth condition disclosed herein. Yeast cells having increased export of 2′FL can then be used to identify endogenous yeast transporters for 2′FL export by identifying endogenous yeast transporters having increased activity in the yeast cells with increased 2′FL export. Endogenous transporters for 2′FL export can be identified by any method known in the art. One such method would be to conduct RNA transcript analysis by one of a variety of techniques, including RNASeq, to look for genes whose transcription is enhanced in a yeast cell where 2′FL export is increased relative to the transcription of the genes in a comparison cell. Another approach is to conduct an analogous proteomics experiment to determine proteins whose biosynthesis is enhanced in a yeast cell where 2′FL export is increased. Those proteins whose genes show enhanced RNA translation in cells with increased export of 2′FL or whose biosynthesis is enhanced in cells having increased export of 2′FL are candidates for endogenous yeast 2′FL transporters. Identified RNA transcripts or proteins with increased activity may be filtered by, for example, analyzing their sequences for similarity to membrane proteins, including known transporters. Once endogenous yeast transporters for 2′FL are identified, yeast cells can be genetically modified to increase the activity of the transporters.

In that regard, in a further aspect, the disclosure provides a genetically engineered microbial cell comprising a genetic modification which increases the activity of one or more endogenous transporters. The activity of the one or more endogenous transporters is increased such that export of 2′FL from the microbial cell is increased.

The genetically engineered microbial cell can be any microbial cell that can be genetically engineered with an increased activity in an endogenous transporter such that export of 2′FL from the microbial cell is increased. Such cells, include, but are not limited to, any bacterial or fungal cell disclosed herein, including any yeast cell disclosed herein. The microbial cell that is genetically engineered to increase the activity of the endogenous transporter may contain further genetic modifications, including, but not limited to, genetic modifications which introduce one or more pathway genes for the production of 2′FL, such as the pathway genes disclosed herein.

The genetic modification which increases the activity of one or more endogenous transporters can increase the activity of any endogenous microbial transporter for 2′FL. In certain embodiments, the genetic modification increases the activity of an endogenous transporter identified by a method for identifying endogenous 2′FL transporters disclosed herein.

Increased activity of an endogenous gene may be achieved by any method known in the art. Methods that are typically known to one skilled in the art include, for example, replacing the promoter of the endogenous gene with a more highly active promoter, replacing the terminator with one that allows greater translation, introducing a chimeric gene encoding the protein encoded by the endogenous gene into the cell on a multi-copy plasmid, introducing a chimeric gene encoding the protein encoded by the endogenous gene into the cell where the promoter of the chimeric gene is of high activity, and/or integrating multiple copies of the endogenous gene and/or of one or more chimeric genes for expression of the coding region of the endogenous gene. In addition, a chimeric gene containing a heterologous coding region for a protein having the same activity as the endogenous gene may be introduced into the microbial cell.

In various embodiments, further genetic engineering modifications are made to the microbial cell to improve the efficiency of production of 2′FL. In certain embodiments, modifications are made to improve carbon flow through the introduced pathway for 2′FL production which may include, but are not limited to, knocking out pathways that compete for key intermediates of the 2′FL pathway and/or redirecting reducing equivalents to the 2′FL pathway. 2′FL exported from a microbial cell as disclosed herein can be isolated from fermentation broth and used in various food products, such as nutritional supplements. For example, the 2′FL can be added to formula for infants, toddlers, or children.

### EXAMPLES

The disclosure is further defined in the following Examples. It should be understood that these Examples, while indicating preferred embodiments, are given by way of illustration only. From the above discussion and these Examples, one skilled in the art can ascertain the characteristics of this disclosure, and without departing from the spirit and scope thereof, can make various changes and modifications to adapt the disclosure to various uses and conditions.

### General Methods

The meaning of abbreviations is as follows: “kb” means kilobase(s), “bp” means base pairs, “nt” means nucleotide(s), “hr” means hour(s), “min” means minute(s), “sec” means second(s), “d” means day(s), “L” means liter(s), “ml” or “mL” means milliliter(s), “μL” means microliter(s), “μg” means microgram(s), “ng” means nanogram(s), “mg” means milligram(s), “mM” means millimolar, “μM” means micromolar, “nm” means nanometer(s), “μmol” means micromole(s), “pmol” means picomole(s),

Standard recombinant DNA and molecular cloning techniques used here are well known in the art and are described by Sambrook, J., Fritsch, E. F. and Maniatis, T., Molecular Cloning: A Laboratory Manual, 2^{nd} ed., Cold Spring Harbor Laboratory: Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory: Cold Spring Harbor, N.Y. (1984); and by Ausubel, F. M. et al., Current Protocols in Molecular Biology, published by Greene Publishing Assoc. and Wiley-Interscience, Hoboken, N.J. (1987), and by Methods in Yeast Genetics, 2005, Cold Spring Harbor Laboratory Press, Cold Spring Harbor, N.Y.

### General Methods

Transformation of Saccharomyces cerevisiae Strains

Saccharomyces cerevisiae strains are made competent for transformation via protocols employing lithium acetate and polyethylene glycol (described in Amberg, D. C., Burke, D. J. and Strathern, J. N. Methods in Yeast Genetics: A Cold Spring Harbor Laboratory Course Manual, Cold Spring Harbor Press, 2005). In most cases, a commercial kit is used (e.g. Frozen EZ Yeast Transformation II Kit™, Zymo Research, Irvine, Calif.), though for some lineages a higher efficiency method such as that by Gietz et al. (1992, Nucleic Acids Res. 20(6): 1425) with extension of 42° C. incubation to 40 minutes is used for chromosomal integrations. Integration events are confirmed by PCR. Yeast cells from colonies or patches are introduced directly into PCR reactions (e.g. JumpStart Red Taq) or pretreated with Chelex® resin (BioRad, Hercules, Calif.) prior to PCR as follows. A sterile toothpick is used to transfer approximately one cubic millimeter of cells to 100 μl of 5% Chelex (w/v) suspended in ddH₂O in a 0.2 ml PCR tube. Tubes are incubated at 99° C. for 10 min followed centrifugation for 3 min at 14000 rpm to pellet all cellular debris at the bottom of the tube.

### Example 1

### Construction of the 2′fucosyllactose-Producing Saccharomyces cerevisiae Strain HS0007

### Integration of Lactose Permease Gene

A nucleic acid molecule having the coding sequence for the lactose permease from Kluyveromyces lactis (LAC12) was obtained from a commercial gene synthesis company (IDT, Coralville, Iowa)(SEQ ID No:1). The linear fragment was cloned into pCRII-Blunt (TOPO) vector (Zero Blunt TOPO cloning vector, Invitrogen) per the manufacturer's instructions. Clones were sequenced. The LAC12 coding region was PCR amplified using primers H89 and H94 (SEQ ID NOs:2 and 3), and this nucleic acid fragment was joined to promoter and terminator sequences using PCR. The PMA1 promoter (SEQ ID NO:4) was amplified from S. cerevisiae genomic DNA using primers H92 and H93 (SEQ ID NOs:5 and 6). The TPS1 terminator (SEQ ID NO:7) was amplified from S. cerevisiae genomic DNA using primers H90 and H91 (SEQ ID NOs:8 and 9). The fused promoter, coding region and terminator were amplified with H91 and H92 primers, digested with BamHI and PmeI, and cloned into pUC19-URA3-YPRCΔ15 (SEQ ID NO:10; described in US Patent Application Publication No. 20130203138, which is incorporated herein by reference for the disclosure of the construction of the plasmid), previously digested with BamHI and PmeI. Ligation mixtures were transformed into E. coli StbI3 cells (Life Technologies). Colonies arising with ampicillin selection (100 μg/mL) were screened by PCR to confirm the LAC12 clones. Positive clones were sequenced. A clone containing a confirmed sequence was linearized with SphI and transformed into strain PNY1500 (also called BP857; described in U.S. Pat. No. 8,871,488) which is a ura34 his34 variant of CEN.PK 113-7D. Cells were plated on synthetic complete medium without uracil. Colonies were screened for the expected integration event using primers BK1042 and H95 for the 5′ end (SEQ ID NOs:11 and 12), and BK1043 and 92 for the 3′ end, (SEQ ID NOs:13 and 14). Two clones were selected for marker recycling, as follows. Clones were grown overnight in yeast extract-peptone-dextrose (YPD) medium, and then streaked onto synthetic complete medium containing 0.1% 5-fluoroorotic acid (5-FOA). Colonies were patched to synthetic complete medium without uracil to confirm lack of growth without uracil (i.e. loss of the URA3 auxotrophic marker). Uracil auxotrophic clones were evaluated by PCR (using primers BK1043 and H96, SEQ ID NO:15) to confirm that the URA3 marker was removed via homologous recombination. Multiple clones were tested for lactose consumption upon transformation with the pHR81-LAC4 plasmid described below. One clone that was able to grow on lactose was designated HS0003.

Beta-galactosidase is temporarily expressed to test for lactose permease activity. The coding sequence for beta-galactosidase from Kluyveromyces lactis (SEQ ID NO:16) was obtained from a commercial gene synthesis company (IDT, Coralville, Iowa). Due to its size, the coding sequence was ordered in two overlapping nucleic acid fragments (5′ fragment and 3′fragment: SEQ ID NOs:17 and 18, respectively). The linear fragments were each cloned into pCRII-Blunt (TOPO) vector (Zero Blunt TOPO cloning vector, Invitrogen) per the manufacturer's instructions. Clones were sequenced. One clone for each plasmid was selected and the two gene fragments were amplified by PCR with primers (H98 and M13ForTOPO for the 5′ fragment and M13RevTOPO and H99 for the 3′ fragment, SEQ ID NOs:19-22). An expression plasmid was assembled using gap repair cloning methodology as follows. The gene fragments were combined with PmeI digested pHR81-ILV5p-R8B2y2 (SEQ ID NO:23; described in US20130252296), which contains the ILV5 promoter and terminator (SEQ ID NOs:24 and 25), and transformed into PNY1500 yprΔ15Δ::LAC12 cells (described above). Transformants were obtained via selection on synthetic complete medium lacking uracil. Colonies were subsequently patched to medium containing lactose as the carbon source. Proper assembly of the expression plasmid (named pHR81::ILV5p-LAC4-ILV5t) was also confirmed using PCR and correlated with the ability to grow on lactose.

### Construction of Plasmids Encoding GDP-Mannose Dehydratase and GDP-4-keto-6-deoxymannose Epimerase Reductase

Nucleic acid molecules having the coding sequences for GDP-mannose dehydratase (GMD) and GDP-4-keto-6-deoxymannose epimerase reductase (GMER) from E. coli were obtained from a commercial gene synthesis company (IDT, Coralville, Iowa) (SEQ ID NOs:26 and 27). The linear gene fragments were cloned into pCRII-Blunt (Zero Blunt TOPO cloning vector, Invitrogen) per the manufacturer's instructions. Clones were sequenced. One clone for each gene was used as a PCR template to add 5′ and 3′ extensions to the genes to allow subsequent cloning by homologous recombination (gap repair cloning). These primers were H17 and H18 (SEQ ID NOs:28 and 29) for GMD and H15 and H16 (SEQ ID NOS:30 and 31) for GMER. The recipient vector was prepared in two fragments from pRS413::BiADH-kivD (described in WO 2014/151645; SEQ ID NO: 98 therein, which is incorporated by reference for the disclosure of the preparation of the plasmid): a 6 kb fragment (PacI/PmeI) and a 2.8 kb fragment (NcoI/EcoRV). The two coding region fragments and the two vector fragments were combined and transformed into PNY1500. Transformants were obtained via selection on synthetic complete medium lacking histidine. The resulting plasmid contained two gene cassettes-one expressing GMD from the PDC1 promoter (SEQ ID NO:32) with the ADH1 terminator (SEQ ID NO:33) and one expressing GMER from a hybrid promoter (PGK1(UAS)-FBA1) (SEQ ID NO:34) with the TDH3 terminator (SEQ ID NO:35). Correct plasmid clones were confirmed by sequencing. One plasmid was designated pRS413::GMD-GMER_Ec.

An additional plasmid expressing GMD and GMER enzymes from Arabidopsis thaliana was also prepared, essentially as described above. The gene sequences (SEQ ID NOs. 36 and 37) were obtained from IDT, cloned and sequenced as described above for the E. coli GMD/GMER pair and then transferred to the yeast expression vector using the same gap repair cloning strategy. The primers used to amplify the genes for this last step were H11 and H12 (GMD_At) and H13 and H14 (GMER_At), corresponding to SEQ ID NOs. 38-41. The host strain for the gap repair cloning was PNY1500 (above). Four clones identified by PCR were subsequently sequenced. One plasmid was designated pRS413::GMD-GMER_At. This plasmid was recovered from yeast cells (Zymo Prep™ Yeast Plasmid Miniprep II kit, Zymo Research, Cat. No. D2004) and propagated in E. coli Stb13 cells (Invitrogen, Cat. No. C7373-03, transformed via the manufacturer's protocol). Plasmid DNA prepared from the transformed Stb13 cells was used to transform yeast strain HS0003. Transformants were selected by plating the transformation mixture on synthetic complete medium without histidine. One clone was designated HS0004.

### Construction of Plasmid Encoding α1,2-fucosyltransferase

A nucleic acid molecule having the coding sequence for a fucosyltransferase (FutC) enzyme from Helicobacter pylori was obtained from a commercial gene synthesis company (IDT, Coralville, Iowa) (SEQ ID NO:42). The linear gene fragment was cloned into pCRII-Blunt (Zero Blunt TOPO cloning vector, Invitrogen) per the manufacturer's instructions. Clones were sequenced using standard M13 forward and reverse primers. One clone was digested with BsaI and the futC coding region fragment was cloned into pY-SUMOstar (Life Sensors, Malvern, Pa.) also previously cut with BsaI. Ligation mixtures were transformed into E. coli Stb13 cells. Colonies arising with ampicillin selection (100 μg/mL) were screened by PCR to confirm the futC clones.

The pY-SUMOstar::futC_Hp plasmid was further modified to change the selectable marker from TRP1 to URA3. This was done by digesting the plasmid with Bsu36I and transforming the linear DNA fragment into HS0004 (above) along with a linear DNA fragment containing the URA3 selectable marker as amplified from pRS426 (ATCC#77107) using primers H305 and H306 (SEQ ID NOs. 43 and 44). Successfully transformed colonies were selected for on synthetic complete medium without uracil and histidine. Colonies were screened by PCR using primers H291 and H292 (SEQ ID NOs. 45 and 46). Three of these transformants were evaluated for production of 2′FL, as described in Example 2. The pY-SUMOstar-URA::futC_Hp plasmid was recovered from one clone (designated HS0006) using the Zymo Prep™ kit. Plasmids were transferred to E. coli Stb13 cells (Invitrogen, catalog number C7373-03) per the manufacturer's instructions. Plasmids prepared from Stb13 cells were used to transform HS0003 along with pRS413::GMD-GMER_Ec (strain and plasmid described above). Transformants again were evaluated as described in Example 2 and one 2′FL-producing clone was designated HS0007. Negative control strains were also prepared by transforming strain HS0003 with only the fucosyltransferase plasmid (plus empty plasmid pRS413) and transforming strain HS0004 with an empty URA3 selectable plasmid (pHR81, ATCC #87541).

### Example 2

### Measurement of 2′FL Production

### Intracellular Measurement of 2′FL

Strains transformed with plasmids carrying 2′FL pathway genes (Example 1) were evaluated in shake flasks. Clones, e.g., HS0007 and siblings, were inoculated into synthetic complete medium without histidine, tryptophan and uracil and incubated at 30° C. with agitation (200 rpm, Infors Multitron platform shaker). Overnight cultures were adjusted to 0.1 to 0.2 OD (Beckman BioPhotomter, Hamburg Germany) and grown to an OD of approximately 1. Lactose was added to 0.5% (w/v) and copper sulfate was added (100 μM) to increase the expression of FutC_Hp, which is under control of the CUP1 promoter. At various times post-induction, culture samples (ca. 2-10 mL) were centrifuged to separate cells from medium. The cell pellets were frozen at −80° C. Culture supernatants were filtered through 0.22 micron Costar Spin-X filter tubes (Corning, Corning, N.Y.) or AcroPrep™ Advance 96 filter plates (Pall, Ann Arbor, Mich.) and stored at −20° C.

Cell pellets were thawed at room temperature just prior to use. An aliquot of 0.425 mL of 0.2 μm filtered NanoPure water was added to each thawed cell pellet, and the pellet was resuspended by pipetting up and down. The suspension was transferred to a 1.5 mL microcentrifuge tube. The sample was heated at 98° C. on a heat block (Eppendorf) for six minutes, cooled briefly on ice, vortexed, and centrifuged at 10,000×g for 10 minutes. An aliquot of 40 μL of the resulting supernatant was added to a new microcentrifuge tube and diluted with the addition of 80 μL of acetonitrile. The 120 μL of acetonitrile-diluted supernatant was transferred to the top of a Nanosep MF Centrifugal Device, 0.2 μm (Pall) which was then centrifuged at 10,000×g for one minute. The filtrate was added to a LC vial with a low volume insert.

Samples were analyzed by UHPLC-ELSD (Shimadzu Nexera X2). The column used was an Acquity UHPLC BEH Amide 1.7 μm, 2.1×100 mm (Waters) with a Waters guard column of the same material. The injection volume for each sample was 4 μL. Buffer A was 10% acetonitrile in water, and Buffer B was 100% acetonitrile. A gradient elution was run that involved an initial hold of 25% Buffer A for 2.3 min, followed by a gradient to 60% Buffer A at 6.5 min, followed by a gradient to 90% Buffer A at 7.00 min and a hold of this percentage to 7.5 min, and then a re-equilibration to 25% Buffer A to 10.0 min. Standard runs with D-(+)-glucose (Sigma-Aldrich G7528 Lot SLBK8673V), α-lactose monohydrate (Carbosynth OL050091401), 2′FL (Carbosynth OF067391403), and lactodifucotetraose (LDFT, Carbosynth OL065671201) resulted in retention times of 1.8 minutes, 3.3 minutes, 4.4 minutes, and 5.4 minutes respectively. Calibration curves were run for these components and were used to produce raw concentration data. OD600 values and the sample amounts were used to normalize the intracellular concentrations as follows:

Normalized mM=(Raw mM)*(0.425 mL+(OD*V_{centrifuged}*0.0009594 mL OD⁻¹))/(OD*V_{centrifuged}*0.0009594 mL OD⁻¹).

The 0.0009594 mL/OD factor was estimated based on a haploid cell volume (Sherman, “Getting started with yeast”, Methods in Enzymology (2002) 350:3-41). Alternatively, data may be normalized to the cell culture volume from which the cells were harvested for comparison to extracellular concentrations. The results are shown in Table 1, below.

TABLE 1
Intracellular 2'FL measurements from shake flask-cultured
cells. 2′FL was extracted from cell pellets and measured as described in
Example 2. Sampling time was 16 hours after addition of lactose (0.5%
w/v) and copper sulfate (0.1 mM).

| Strain | | 2′FL normalized to |
|---|---|---|
| Designation | | intracellular |
| (if | | concentration, |
| applicable) | Base Strain/plasmids | mM |
| | HS0003/pY-SUMOstar- | Not detected |
| | URA:: futC_Hp/pRS413 (3 | |
| | clones) | |
| | HS0003/pHR81/pRS413:: GMD- | Not detected |
| | GMER_At (3 clones) | |
| HS0006 | HS0003/pY-SUMOstar- | 59.5 ± 0.3 (n = 2) |
| | URA:: futC_Hp/pRS413:: GMD- | |
| | GMER_At | |
| | HS0003/pY-SUMOstar- | 101 |
| | URA:: futC_Hp/pRS413:: GMD- | |
| | GMER_Ec Clone #1 | |
| | HS0003/pY-SUMOstar- | 118 |
| | URA:: futC_Hp/pRS413:: GMD- | |
| | GMER_Ec Clone #2 | |
| HS0007 | HS0003/pY-SUMOstar- | 107 |
| | URA:: futC_Hp/pRS413:: GMD- | |
| | GMER_Ec Clone #3 | |

### Extracellular Detection of 2′FL

Extracellular 2′-fucosyl lactose was measured with an enzyme based fluorometric assay. Yeast culture supernatants were filtered using Spin-X 0.22 μM Nylon tube filters and 100 μl of filtrates were diluted two fold into 202 mM sodium phosphate pH 6, containing 1.51 units of T. maritima fucosidase (E-FUC™, Megazyme International Ireland). The mixtures were incubated at 90° C. for 10 minutes. The amounts of fucose in the resultant solutions were measured with the L-fucose assay kit (K-Fucose, Megazyme International Ireland), based on fucose dehydrogenase catalyzed oxidation of fucose with concomitant reduction of NADP. 26.2 μl of fucosidase treated samples were diluted 10 fold in the fucose dehydrogenase reaction mixture, prepared according to the vendor. The solutions were incubated for 19 min at 37° C. NADPH fluorescence was then measured in a Wallac 1420 Victor3 Microplate Reader (Perkin Elmer), employing a 355 nm cut-off filter for excitation and 450 nm filter for emission. A fucose standard was employed to calculate fucose formed in each reaction. Samples with and without fucosidase were compared to specifically quantitate the amounts of fucose generated during the fucosidase treatment step, providing extracellular 2′fucosyllactose concentrations in the supernatants.

### Example 3

### Intracellular and Extracellular Concentrations of 2′FL Under Various Growth Conditions

### Glucose Excess Versus Glucose Limited

### Inoculum Preparation

A frozen vial of HS0007 (prepared as described in Example 1) was thawed and transferred to 10 mL synthetic complete medium with 2% glucose in a 125 mL vented shake flask, and incubated at 30° C. and 300 rpm shaking for several hours. Two seed flasks were prepared using this culture in two 250 mL vented shake flasks with 40 mL of synthetic complete medium with 2% glucose for further growth at 30° C. and 300 rpm shaking. When the culture reached OD600 about 4, the two flask cultures were used to inoculate two 1 L fermenters. The synthetic complete medium composition is as follows: yeast nitrogen base without amino acids (Difco), 6.7 g/L; Synthetic Complete Drop-out:(Kaiser) -his -ura (Formedium, England), 1.8 g/L; glucose was added to 2% (w/v) for the inoculum growth. The pH was adjusted to 5.2 with 20% potassium hydroxide and the medium filter sterilized through a 0.22μ filter.

### Fermenter Preparation and Operation:

Fermentations were carried out in 1 L Biostat B DCU3 fermenters (Sartorius, USA). Two fermenters were prepared with 500 mL 0.9% (w/v) NaCl solution and sterilized at 121′ C for 30 minutes. After cooling, the salt solution was pushed out and 760 mLs medium, which had been previously filter sterilized, was pumped into the fermenters. Synthetic complete medium with 0.2 mL antifoam (DF204, Sigma, USA) was used in both fermentations; the medium for one fermenter (V1) was prepared with 2% glucose and for the second fermenter (V2) with 0.1% glucose. The temperature of the fermenter was maintained at 30° C., and pH controlled at 5.5 with 20% KOH throughout the entire fermentations. Aeration was controlled at 0.4 standard liters per minute, and dissolved oxygen controlled at 20% by agitation. Samples were drawn and analyzed for optical density at 600 nm and for glucose concentration by a YSI Select Biochemisty Analyzer (YSI, Inc., Yellow Springs, Ohio). Glucose excess was maintained throughout the fermentation in V1, at 5-30 g/L by manual additions of a 50% (w/w) solution. Fermenter V2 was run with glucose limitation using a programmed exponential ramp feed of 50% (w/w) glucose controlled with an exponential ramp of 0.12/hr. The glucose feed was delivered via syringe pumps (KD Scientific, Inc., USA). When glucose measurements in Fermenter V2 exceeded 0.1 g/L, the feed rate was slowed to bring it back to the limited condition. When the optical density was about 1.5, CuSO₄ to a final concentration of 100 μM and lactose to a final concentration of 5 g./L were added to each fermenter.

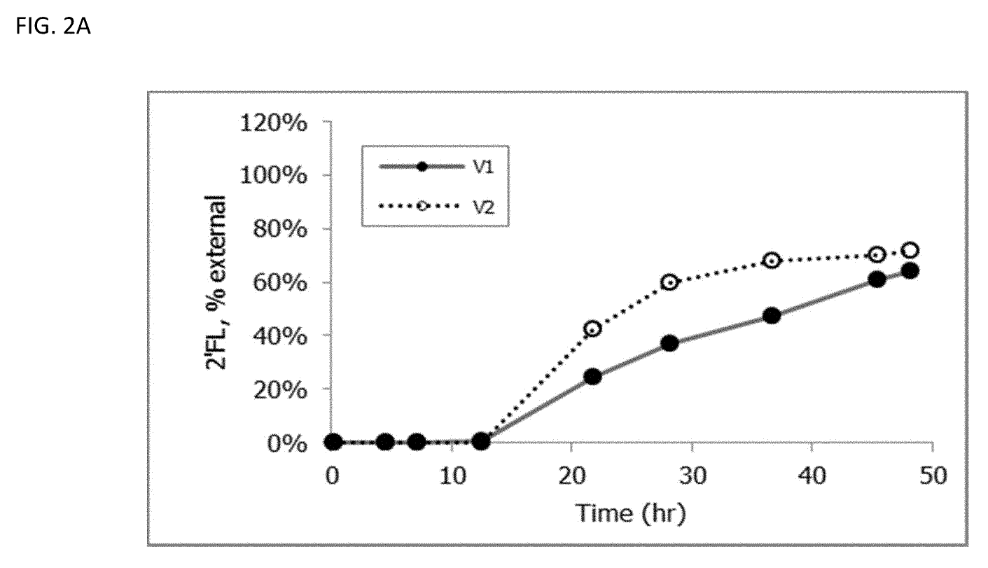

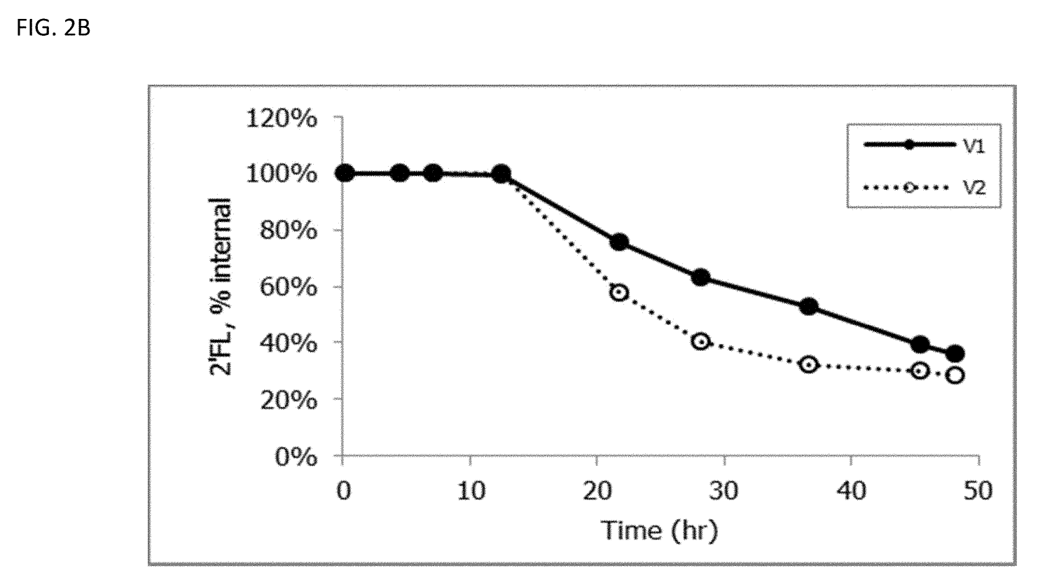

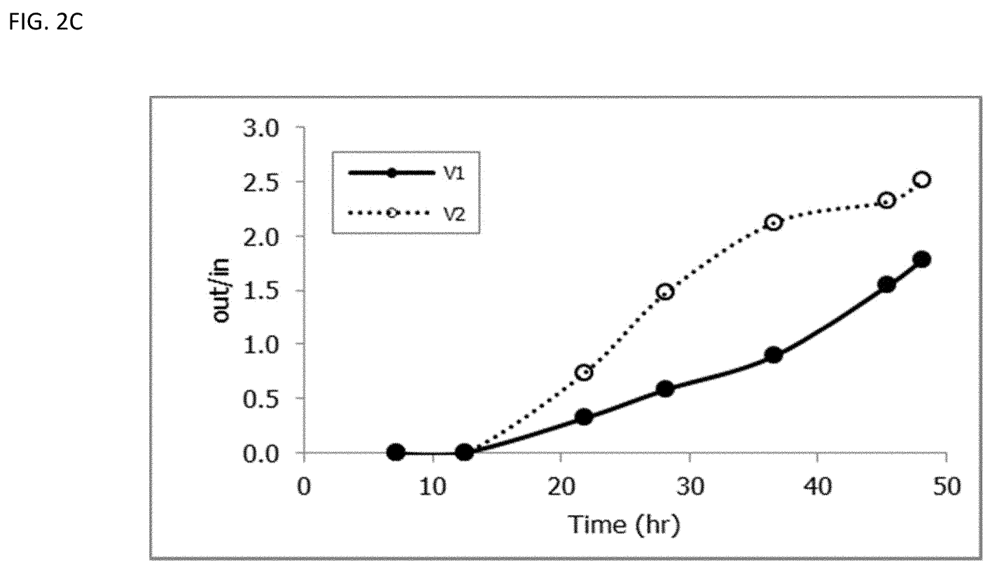

Samples from both fermenters were centrifuged to separate the biomass and cell pellets. Both fractions were stored at −80 C until analysis at the end of the experiment. Intracellular and extracellular 2′FL amounts from the cell pellets were determined as described in Example 2. The results in terms of extracellular and intracellular 2′FL percentages and the extracellular to intracellular 2′FL ratio are shown in FIGS. 2A-2C, respectively. As shown in FIGS. 2A-2C, in cells grown in glucose-limited conditions, a greater amount (percentage and ratio) of the 2′FL was found in the extracellular fraction.

### Glucose Versus Ethanol as a Carbon Source

### Inoculum Preparation

A frozen vial of HS0007 (prepared as described in Example 1) was thawed and transferred to 10 mL synthetic complete medium with 2% glucose in a 125 mL vented shake flask, and incubated at 30° C. and 300 rpm shaking for several hours. Two seed flasks were prepared using this culture in two 250 mL vented shake flasks with 40 mL of synthetic complete medium with 2% glucose for further growth at 30° C. and 300 rpm shaking. When the culture reached OD600 about 4, the two flask cultures were used to inoculate two 1 L fermenters. The synthetic complete medium composition is as follows: yeast nitrogen base without amino acids (Difco), 6.7 g/L; Synthetic Complete Drop-out:(Kaiser) -his -ura (Formedium, England), 1.8 g/L; glucose was added to 2% (w/v) for the inoculum growth. The pH was adjusted to 5.2 with 20% potassium hydroxide and the medium filter sterilized through a 0.22μ filter.

### Fermenter Preparation and Operation:

Fermentations were carried out in 1 L Biostat B DCU3 fermenters (Sartorius, USA). Two fermenters were prepared with 500 mL 0.9% (w/v) NaCl solution and sterilized at 121′ C for 30 minutes. After cooling, the salt solution was pushed out and 760 mLs medium, which had been previously filter sterilized, was pumped into the fermenters. Synthetic complete medium with 0.2 mL antifoam (DF204, Sigma, USA) was used in both fermentations; the medium for one fermenter (V1) was prepared with 2% glucose and for the second fermenter (V2) with 2% ethanol. The temperature of the fermenter was maintained at 30° C., and pH controlled at 5.5 with 20% KOH throughout the entire fermentations. Aeration was controlled at 0.4 standard liters per minute, and dissolved oxygen controlled at 20% by agitation. Samples were drawn and analyzed for optical density at 600 nm and for glucose concentration by a YSI Select Biochemisty Analyzer (YSI, Inc., Yellow Springs, Ohio). Glucose excess was maintained throughout the fermentation in V1, at 5-30 g/L by manual additions of a 50% (w/w) solution. Fermenter V2 was fed ethanol back to 20 g/L at 25 hours elapsed fermentation time. When the optical density was about 1.5, CuSO₄ to a final concentration of 100 μM and lactose to a final concentration of 5 g./L were added to each fermenter.

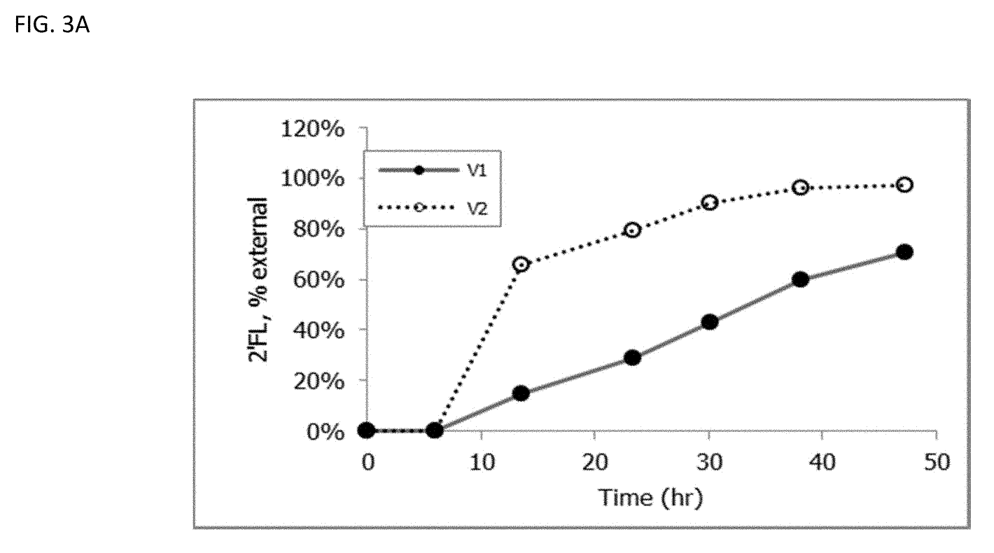

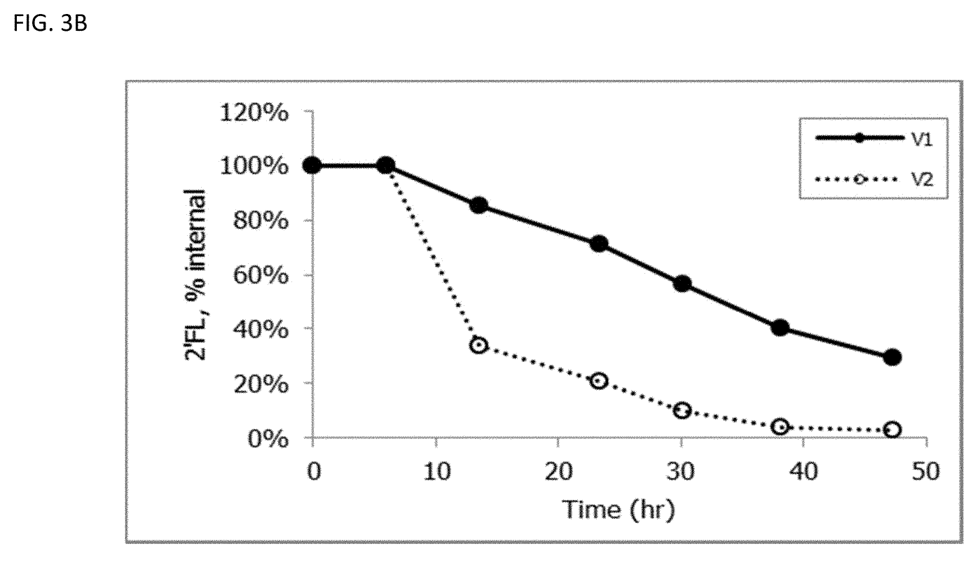

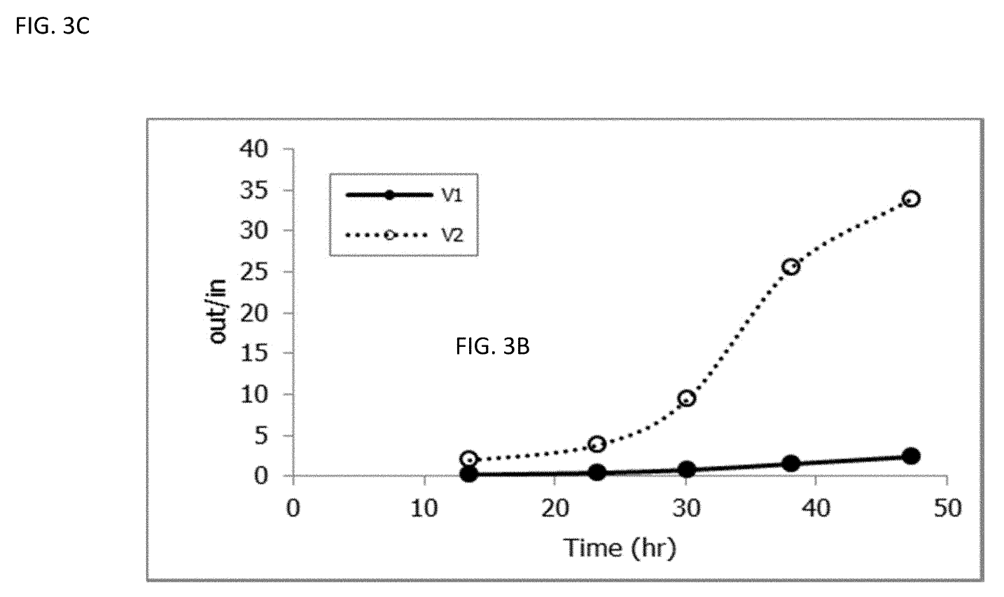

Samples from both fermenters were centrifuged to separate the biomass and cell pellets. Both fractions were stored at −80 C until analysis at the end of the experiment. Intracellular and extracellular 2′FL amounts from the cell pellets were determined as described in Example 2. The results in terms of extracellular and intracellular 2′FL percentages and the extracellular to intracellular 2′FL ratio are shown in FIGS. 3A-3C, respectively. As shown in FIGS. 3A-3C, in cells grown in the presence of ethanol, a greater amount (percentage and ratio) of the 2′FL was found in the extracellular fraction.

### Synthetic Versus Defined Medium

### Inoculum Preparation

A frozen vial of HS0007 (prepared as described in Example 1) was thawed and transferred to 10 mL synthetic complete medium with 2% glucose in a 125 mL vented shake flask, and incubated at 30° C. and 300 rpm shaking for several hours. Two seed flasks were prepared using this culture in two 250 mL vented shake flasks with 40 mL of synthetic complete medium with 2% glucose for further growth at 30° C. and 300 rpm shaking. When the culture reached OD600 about 4, the two flask cultures were used to inoculate two 1 L fermenters. The synthetic complete medium composition is as follows: yeast nitrogen base without amino acids (Difco), 6.7 g/L; Synthetic Complete Drop-out:(Kaiser) -his -ura (Formedium, England), 1.8 g/L; glucose was added to 2% (w/v) for the inoculum growth. The pH was adjusted to 5.2 with 20% potassium hydroxide and the medium filter sterilized through a 0.22μ filter.

### Fermenter Preparation and Operation:

Fermentations were carried out in 1 L Biostat B DCU3 fermenters (Sartorius, USA). Two fermenters were prepared with 500 mL 0.9% (w/v) NaCl solution and sterilized at 121′ C for 30 minutes. After cooling, the salt solution was pushed out and 760 mLs medium, which had been previously filter sterilized, was pumped into the fermenters. Synthetic complete medium with 2% glucose and 0.2 mL antifoam (DF204, Sigma, USA) was used in fermenter V1. The second fermenter, V2, was prepared with a minimal medium with the following composition, per liter: 5 g ammonium sulfate, 6 g potassium phosphate monobasic, 2 g magnesium sulfate heptahydrate, 1 mL of a trace mineral solution (prepared in 1 L water: 15 g EDTA, 4.5 g zinc sulfate heptahydrate, 0.8 g manganese chloride dehydrate, 0.3 g cobalt chloride hexahydrate, 0.3 g copper sulfate pentahydrate, 0.4 g disodium molybdenum dehydrate, 4.5 g calcium chloride dihydrate, 3 g iron sulfate heptahydrate, 1 g boric acid, 0.1 g potassium iodide) and 1 mL of a vitamin mixture (in 1 L water, 50 mg biotin, 1 g Ca-pantothenate, 1 g nicotinic acid, 25 g myo-inositol, 1 g pyridoxol hydrochloride, 0.2 g p-aminobenzoic acid), 20 g glucose and 0.2 mL Sigma Antifoam 204.

The temperature of the fermenters was maintained at 30° C., and pH controlled at 5.5 with 20% KOH throughout the entire fermentations. Aeration was controlled at 0.4 standard liters per minute, and dissolved oxygen controlled at 20% by agitation. Samples were drawn and analyzed for optical density at 600 nm and for glucose concentration by a YSI Select Biochemistry Analyzer (YSI, Inc., Yellow Springs, Ohio). Glucose excess was maintained throughout both fermentations, at 5-30 g/L, by manual additions of a 50% (w/w) solution. When the optical density was about 1.5, CuSO₄ to a final concentration of 100 μM and lactose to a final concentration of 5 g./L were added to each fermenter.

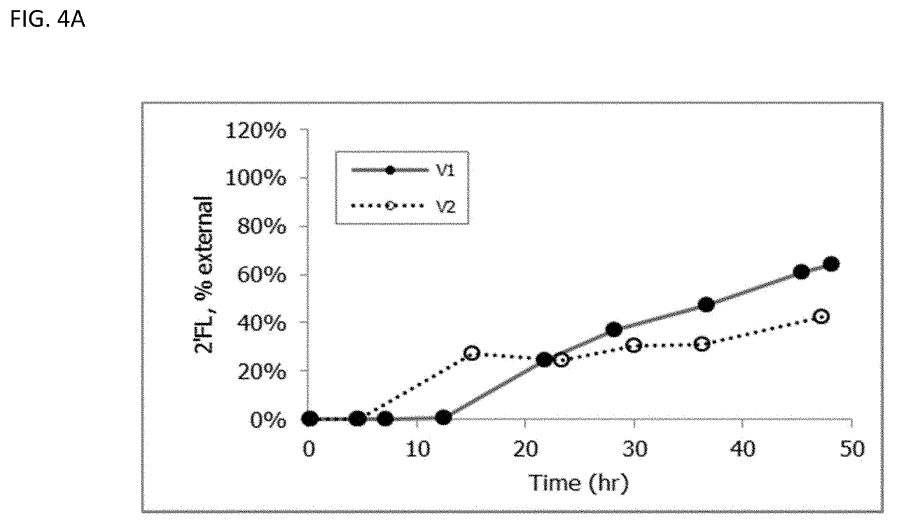

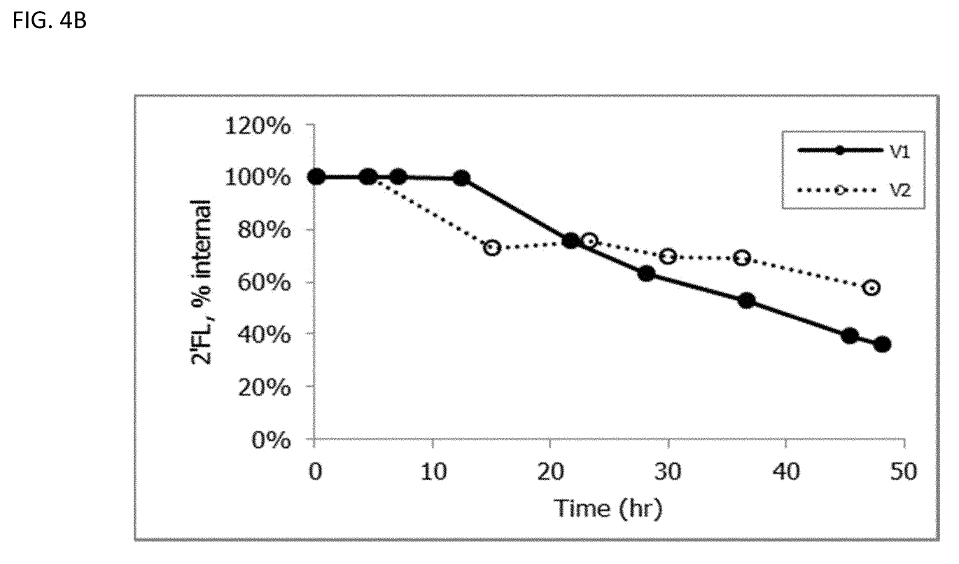

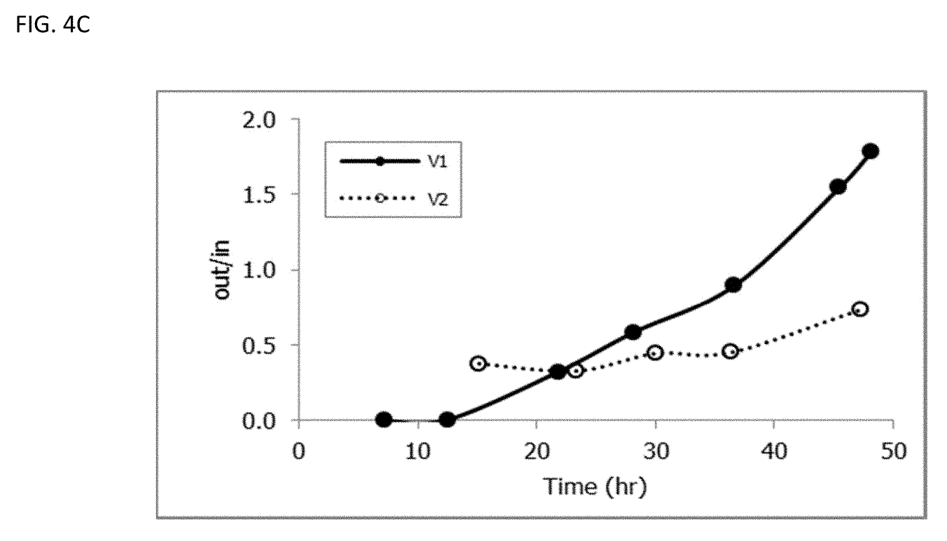

Samples from both fermenters were centrifuged to separate the biomass and cell pellets. Both fractions were stored at −80 C until analysis at the end of the experiment. Intracellular and extracellular 2′FL amounts from the cell pellets were determined as described in Example 2. The results in terms of extracellular and intracellular 2′FL percentages and the extracellular to intracellular 2′FL ratio are shown in FIGS. 4A-4C, respectively. As shown in FIGS. 4A-4C, in cells grown in the presence of synthetic complete medium, a greater amount (percentage and ratio) of the 2′FL was found in the extracellular fraction.

### Example 4

### Identification of Endogenous Transporters for 2′FL

The following example discloses a method to identify 2′FL transporters endogenous to microbial cells.

Samples are taken over the course of the fermentations described in Example 3 for transcriptome analysis. A 1 mL sample is taken directly from the fermenter and centrifuged for 2 minutes. After removal of the supernatant, 1 mL of Trizol® Reagent (Life Technologies, USA) is added to the tube, the pellet resuspended, and samples stored at −80 C until analysis. The samples are processed to recover RNA submitted for RNASeq analysis. Potential candidates for endogenous transporters are identified by comparison of the transcript profile of cells grown in the fermentation conditions described in Example 3. Genes that are more highly expressed in cells grown in each of the respective comparative conditions where increased 2′FL export was seen (e.g., ethanol versus glucose as the carbon source) are further analyzed to select those genes that are both more highly expressed and that are either annotated as coding for transporters or are homologous to known transporters. The selected candidates are then evaluated by knocking out the gene and by overexpressing the gene in yeast by a constitutive promoter. The yeast cells containing knockouts and overexpressing the candidate transporters are then assayed for loss and gain of 2′FL export function, respectively.

What is claimed is:

1. A fermentation broth comprising a microbial cell having increased export of 2′ fucosyllactose, the microbial cell having been subjected to a condition under which the activity of an endogenous transporter is increased relative to the activity of the endogenous transporter in the microbial cell in the absence of subjecting the microbial cell to the condition.

2. The fermentation broth of claim 1 wherein the condition comprises one or more of growing the microbial cell in a medium comprising amino acids, growing the microbial cell in a glucose limited medium, and growing the microbial cell in a medium comprising ethanol.

3. The fermentation broth of claim 1 wherein the microbial cell is a yeast cell.

4. A method for increasing the export of 2′ fucosyllactose from a microbial cell comprising:
a) obtaining a 2′FL-containing microbial cell; and
b) subjecting the microbial cell to a condition under which the activity of an endogenous transporter is increased relative to the activity of the endogenous transporter in the microbial cell in the absence of subjecting the microbial cell to the condition.

5. The method of claim 4 wherein subjecting the microbial cell to a condition under which the activity of an endogenous transporter is increased comprises one or more of growing the microbial cell in a medium comprising amino acids, growing the microbial cell in a glucose limited medium, and growing the microbial cell in a medium comprising ethanol.

6. The method of claim 4 wherein the microbial cell is a yeast cell.

7. A method of identifying an endogenous yeast transporter for exporting 2′ fucosyllactose from a yeast cell, the method comprising:
a) obtaining a 2′FL-containing yeast cell;
b) subjecting the yeast cell to a condition under which the export of 2′ fucosyllactose is increased relative to the export of 2′ fucosyllactose in the yeast cell in the absence of subjecting the yeast cell to the condition; and
c) identifying an endogenous yeast transporter with increased activity in the yeast cell as an endogenous yeast transporter for exporting 2′ fucosyllactose.

8. The method of claim 7 wherein subjecting the yeast cell to a condition under which the export of 2′ fucosyllactose is increased comprises one or more of growing the yeast cell in a medium comprising amino acids, growing the yeast cell in a glucose limited medium, and growing the yeast cell in a medium comprising ethanol.

9. A genetically engineered microbial cell comprising a genetic modification which increases the activity of an endogenous transporter whereby export of 2′ fucosyllactose from the microbial cell is increased.

10. The genetically engineered microbial cell of claim 9 wherein the microbial cell is a yeast cell.

11. The genetically engineered yeast cell of claim 10 wherein the genetic modification increases the activity of an endogenous transporter identified by the method of claim 7 or 8.


SEQUENCE LISTING (46 sequences)

SEQ ID NO: 1 - DNA, 1764 bp, Kluyveromyces lactis
atggccgaccactcaagttccagctcttctttgcaaaagaaaccaatcaacaccatcgaacacaaggatactttgggaaacgacagagatcacaaggaagccttgaactccgacaacgacaatacttctggcttgaagattaacggtgttccaatcgaggatgccagagaagaagtcttgttacctggttacctatccaagcaatactacaaattgtacggtttatgtttcatcacttacttgtgtgctaccatgcaaggatacgatggtgctttgatgggctctatctacaccgaagacgcttacttaaagtactaccacttggatatcaactccagttctggtaccggcttggttttctccattttcaacgttggtcaaatctgtggagctttctttgtcccattgatggactggaagggcagaaaaccagctatcctaattggttgtttgggcgttgtcattggtgctatcatcagttctttgactacaaccaagtctgctttgatcggtggcagatggtttgttgctttcttcgccactattgccaacgctgccgctccaacttactgtgccgaagttgctccagctcacttgcgtggcaaggttgctggtttgtacaacaccttatggtccgtcggttctattgttgctgccttctctacttacggtaccaacaagaatttcccaaactcttccaaggctttcaaaatccctttgtacttacaaatgatgttcccaggtttggtttgcattttcggttggttgatcccagaatctcccagatggttggtcggtgttggaagggaggaagaagccagagagttcatcattaagtaccacttgaacggcgacagaacccacccattgctggacatggagatggccgaaatcattgagtctttccacggtaccgacttgtccaacccactggaaatgttggacgttcgttctctattcagaaccaggtccgaccgttacagagctatgttggtcatcttaatggcctggtttggtcaattctctggaaacaatgtttgttcttactacttaccaactatgttgagaaacgttggtatgaagtccgtctctctaaacgtattgatgaatggtgtctactctatcgttacctggatatcttccatttgtggtgctttctttatcgacaagattggcaggagagaaggtttcttgggctccatctctggtgctgccttggctttaactggtctatctatctgtactgccagatacgaaaagaccaagaaaaagtctgcctccaacggtgctttggttttcatctacctatttggtggaatcttctcttttgctttcactccaatgcaatctatgtactccaccgaagtctctacaaacttgaccagatcaaaggctcaattgttaaacttcgttgtctctggtgtggctcaattcgtcaaccaatttgccactccaaaggctatgaaaaacatcaagtactggttttacgttttctacgtcttctttgacatcttcgagttcattgtcatctactttttcttcgtcgaaaccaagggcagatccctggaagaattggaggttgtcttcgaagctccaaacccgagaaaggcttctgtcgatcaagccttcttggctcaagtcagagccactttggttcaaagaaacgacgtccgtgttgccaatgctcaaaacttgaaggagcaagaaccattgaagtccgacgccgatcatgtcgaaaagttgtccgaggccgaatctgtttag

SEQ ID NO: 2 - DNA, 30 bp, artificial sequence
  H89 primer
ctaaacagattcggcctcggacaacttttc

SEQ ID NO: 3 - DNA, 60 bp, artificial sequence
  H94 primer
agaaagatttaattatcaaacaatatcaatatggccgaccactcaagttccagctcttct

SEQ ID NO: 4 - DNA, 470 bp, Saccharomyces cerevisiae
tttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagctagttaaagaaaatcattgaaaagaataagaagataagaaagatttaattatcaaacaatatcaat

SEQ ID NO: 5 - DNA, 42 bp, artificial sequence
  H92 primer
gcaaaggatcctttttacctctgtggaaattgttactctcac

SEQ ID NO: 6 - DNA, 30 bp, artificial sequence
  H93 primer
attgatattgtttgataattaaatctttct

SEQ ID NO: 7 - DNA, 250 bp, Saccharomyces cerevisiae
tgaacccgatgcaaatgagacgatcgtctattcctggtccggttttctctgccctctcttctattcactttttttatactttatataaaattatataaatgacataactgaaacgccacacgtcctctcctattcgttaacgcctgtctgtagcgctgttactgaagctgcgcaagtagttttttcaccgtataggccctctttttctctctctttctttctctcccgcgctgatctcttcttcgaaaca

SEQ ID NO: 8 - DNA, 60 bp, artificial sequence
  H90 primer
gaaaagttgtccgaggccgaatctgtttagtgaacccgatgcaaatgagacgatcgtcta

SEQ ID NO: 9 - DNA, 44 bp, artificial sequence
  H91 primer
tccattgtttaaactgtttcgaagaagagatcagcgcgggagag

SEQ ID NO: 10 - DNA, 4827 bp, artificial sequence
  pUC19-URA3-YPRC plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccccaaaaggaatattgggtcagatgaatggacgcgaatgcaagacagaagtccaaatcacgtcaagacaaagaaagaaagaaagaaaaactaacacattaatgtagttttaaaatttcaaatccgaacaacagagcatagggtttcgcaaaggatccggcgcgccgtttaaacaatggaaggtcgggatgagcatatacaagcactaagaagaacaatacagaactctacacggtattattgtgctacaagctcgagtaaaaccgagtgttttgacgatactaacgttgttaagaaagtaacttgttatcaaactcattaccaacttgtgattaattggtgaataatatgataattgtcgaaattccattgttggtaaagcctataatattatgtatacagattatactagaaattctctcgagaatataagaatccccaaaattgaatcggtatttctacatactaatattaccattacttctcctttcgttttatatgtttcattcctattacattatcgatctttgcatttcagcttccattatatttgatgtctgttttatgtccccacgttacaccgcatgtgacagtatactagtaacatgagtgctaccgaatagatgacattttagactttcattccaacaacttggttgacagaatgttacgtaggccggccaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaagcttcatggccttgcggccgcttcatatatgacgtaataaaatgaaatgtagattcattttgtagattcctatatcctcagagagaatttttggtatatcctatatgcataatattatagtctttgccaacaatcgaaaccaaacatatatcttaaaatacaccactttctcaaataaattcgttaaataacggtgtgttgaaatgtttaccgtaacttgtaacagctctaacaactcatacctgctatgtactgattccaagaaaaaaaattaattaatctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 11 - DNA, 20 bp, artificial sequence
  BK1042 primer
tcttaaaatgctcttcttat

SEQ ID NO: 12 - DNA, 20 bp, artificial sequence
  H95 primer
cgaaactaatggaggggagc

SEQ ID NO: 13 - DNA, 20 bp, artificial sequence
  BK1043 primer
acaatggaatttcgacaatt

SEQ ID NO: 14 - DNA, 22 bp, artificial sequence
  92 primer
gagaagatgcggccagcaaaac

SEQ ID NO: 15 - DNA, 20 bp, artificial sequence
  H96 primer
taacgcctgtctgtagcgct

SEQ ID NO: 16 - DNA, 3078 bp, Kluyveromyces lactis
atgtcttgtttgatcccagaaaacctaagaaatccaaagaaagttcacgaaaacagattgccaaccagggcttactactacgaccaagatatcttcgaatccttaaacggtccttgggcttttgccttattcgacgctcccttggatgctccagacgccaagaacttggattgggaaactgccaagaaatggtctactatttccgttccatctcactgggaactacaagaggactggaaatacggcaagccaatctacaccaacgtccaatacccaatacctatcgacattccaaaccctccgaccgtcaatccaactggtgtttacgctcgtactttcgaattggactccaagtctatcgaaagtttcgagcacagattgcgtttcgaaggtgtcgataactgttacgaattgtacgtcaacggtcaatacgttggcttcaacaagggttccagaaatggagccgaattcgatatacaaaagtacgtttccgagggcgaaaacttggttgttgtcaaagtgttcaagtggtccgactctacttacatcgaagaccaagatcaatggtggttatctggtatctacagagacgtctctttgttaaagctaccaaagaaagctcacatcgaagacgtcagagttacaaccactttcgtcgattctcaataccaagacgccgaattgtctgttaaggtggatgtccaaggcagttcttacgatcacatcaacttcaccttgtacgagccagaagacggttccaaagtttacgacgcttcttccttgctaaacgaagagaatggcaacaccactttttctacaaaggaattcatttccttttctaccaagaaaaacgaagagactgctttcaagatcaacgtcaaagctccagaacactggactgccgagaacccaaccttgtacaagtaccaactggatttgattggctccgacggttcagttattcaatctatcaagcaccatgtcggtttcagacaagtggaattgaaggatggcaatatcaccgtcaacggcaaggacatcttgttccgtggtgtcaacagacacgaccaccatccacgtttcggcagagctgtcccattggatttcgttgtcagagacttaatcttgatgaagaaattcaacatcaatgctgtcagaaactctcattacccaaatcaccccaaggtttacgatttgttcgacaagctgggtttttgggttatcgacgaagccgatttggagacccacggtgttcaagaaccattcaacagacacaccaatttggaagccgagtacccagacaccaagaacaaattgtacgacgtcaacgctcactacttgtccgacaacccagaatacgaggttgcttacttggacagagcctctcaattggttttacgtgacgtcaaccacccatccatcatcatttggtctttgggcaacgaagcctgttacggcagaaaccacaaggctatgtacaaactaatcaagcaactggacccaaccagattggttcactacgaaggcgacttaaacgctttgtctgccgacatcttctcctttatgtacccaaccttcgaaatcatggagcgttggagaaagaatcacaccgacgaaaacggcaagttcgagaaaccattgattctatgcgaatacggtcacgctatgggcaacggaccaggttctttgaaagagtaccaagaattgttttacaaagagaagttctaccaaggtggctttatctgggaatgggccaaccacggtatcgaattcgaggacgtttctactgccgatggcaagttgcacaaagcctacgcttacggtggcgatttcaaggaagaggtccacgacggtgttttcattatggacggtttgtgcaacagcgaacacaatccaactcctggtttggtcgaatacaagaaagttatcgaaccagtccacatcaagattgctcatggttctgttactatcaccaacaagcacgatttcattactacagaccacttgttattcatcgacaaagatactggcaagaccatcgacgttccgtctttgaagccagaagagtctgttaccattccatccgatactacatacgttgtcgctgtgttgaaggacgatgccggtgttttgaaggctggccacgaaattgcttggggtcaagccgaactacctttgaaggtcccagacttcgttaccgaaactgccgagaaagccgctaagatcaacgatggaaaaagatacgtttccgtcgaatcttcaggtttacacttcatcctggacaagttattgggcaaaatcgaatctttgaaggtcaagggaaaagaaatctcttccaagttcgaaggttcttcaattactttctggagaccacctaccaacaacgacgaaccaagagatttcaagaactggaagaaatacaatatcgacttgatgaagcaaaacattcacggagtttctgtcgaaaagggttccaacggctctttggctgtggttactgtcaacagcagaatttctccagttgtcttttactacggtttcgaaactgtccaaaagtacaccattttcgccaacaagatcaatttgaacacctctatgaagttaactggcgaataccaacctcccgacttcccaagagttggttacgaattctggttgggcgactcttacgaatccttcgagtggttgggcagaggtcctggagaatcttacccagacaaaaaggaatctcaaagattcggtttgtacgattccaaggacgtcgaggaattcgtttacgattacccacaagaaaacggaaatcacaccgatactcatttcttgaacatcaagtttgaaggtgctggcaagttatctatcttccaaaaggaaaaaccattcaacttcaagatttccgacgagtacggtgtcgatgaagccgctcacgcttgcgacgtcaagagatacggtcgtcactacctaagattggaccatgctattcacggtgttggctccgaagcctgtggtccagctgttttggaccaatacagactaaaggctcaagacttcaacttcgaatttgacttggctttcgaatag

SEQ ID NO: 17 - DNA, 1700 bp, artificial sequence
  5′ end of beta-galactosidase coding region from Kluyveromyces lactis
atgtcttgtttgatcccagaaaacctaagaaatccaaagaaagttcacgaaaacagattgccaaccagggcttactactacgaccaagatatcttcgaatccttaaacggtccttgggcttttgccttattcgacgctcccttggatgctccagacgccaagaacttggattgggaaactgccaagaaatggtctactatttccgttccatctcactgggaactacaagaggactggaaatacggcaagccaatctacaccaacgtccaatacccaatacctatcgacattccaaaccctccgaccgtcaatccaactggtgtttacgctcgtactttcgaattggactccaagtctatcgaaagtttcgagcacagattgcgtttcgaaggtgtcgataactgttacgaattgtacgtcaacggtcaatacgttggcttcaacaagggttccagaaatggagccgaattcgatatacaaaagtacgtttccgagggcgaaaacttggttgttgtcaaagtgttcaagtggtccgactctacttacatcgaagaccaagatcaatggtggttatctggtatctacagagacgtctctttgttaaagctaccaaagaaagctcacatcgaagacgtcagagttacaaccactttcgtcgattctcaataccaagacgccgaattgtctgttaaggtggatgtccaaggcagttcttacgatcacatcaacttcaccttgtacgagccagaagacggttccaaagtttacgacgcttcttccttgctaaacgaagagaatggcaacaccactttttctacaaaggaattcatttccttttctaccaagaaaaacgaagagactgctttcaagatcaacgtcaaagctccagaacactggactgccgagaacccaaccttgtacaagtaccaactggatttgattggctccgacggttcagttattcaatctatcaagcaccatgtcggtttcagacaagtggaattgaaggatggcaatatcaccgtcaacggcaaggacatcttgttccgtggtgtcaacagacacgaccaccatccacgtttcggcagagctgtcccattggatttcgttgtcagagacttaatcttgatgaagaaattcaacatcaatgctgtcagaaactctcattacccaaatcaccccaaggtttacgatttgttcgacaagctgggtttttgggttatcgacgaagccgatttggagacccacggtgttcaagaaccattcaacagacacaccaatttggaagccgagtacccagacaccaagaacaaattgtacgacgtcaacgctcactacttgtccgacaacccagaatacgaggttgcttacttggacagagcctctcaattggttttacgtgacgtcaaccacccatccatcatcatttggtctttgggcaacgaagcctgttacggcagaaaccacaaggctatgtacaaactaatcaagcaactggacccaaccagattggttcactacgaaggcgacttaaacgctttgtctgccgacatcttctcctttatgtacccaaccttcgaaatcatggagcgttggagaaagaatcacaccgacgaaaacggcaagttcgagaaaccattgattctatgcgaatacggtcacgctatgggcaacggaccaggttctttgaaagagtacca

SEQ ID NO: 18 - DNA, 1438 bp, artificial sequence
  3′ end of beta-galactosidase coding region from Kluyveromyces lactis
gattctatgcgaatacggtcacgctatgggcaacggaccaggttctttgaaagagtaccaagaattgttttacaaagagaagttctaccaaggtggctttatctgggaatgggccaaccacggtatcgaattcgaggacgtttctactgccgatggcaagttgcacaaagcctacgcttacggtggcgatttcaaggaagaggtccacgacggtgttttcattatggacggtttgtgcaacagcgaacacaatccaactcctggtttggtcgaatacaagaaagttatcgaaccagtccacatcaagattgctcatggttctgttactatcaccaacaagcacgatttcattactacagaccacttgttattcatcgacaaagatactggcaagaccatcgacgttccgtctttgaagccagaagagtctgttaccattccatccgatactacatacgttgtcgctgtgttgaaggacgatgccggtgttttgaaggctggccacgaaattgcttggggtcaagccgaactacctttgaaggtcccagacttcgttaccgaaactgccgagaaagccgctaagatcaacgatggaaaaagatacgtttccgtcgaatcttcaggtttacacttcatcctggacaagttattgggcaaaatcgaatctttgaaggtcaagggaaaagaaatctcttccaagttcgaaggttcttcaattactttctggagaccacctaccaacaacgacgaaccaagagatttcaagaactggaagaaatacaatatcgacttgatgaagcaaaacattcacggagtttctgtcgaaaagggttccaacggctctttggctgtggttactgtcaacagcagaatttctccagttgtcttttactacggtttcgaaactgtccaaaagtacaccattttcgccaacaagatcaatttgaacacctctatgaagttaactggcgaataccaacctcccgacttcccaagagttggttacgaattctggttgggcgactcttacgaatccttcgagtggttgggcagaggtcctggagaatcttacccagacaaaaaggaatctcaaagattcggtttgtacgattccaaggacgtcgaggaattcgtttacgattacccacaagaaaacggaaatcacaccgatactcatttcttgaacatcaagtttgaaggtgctggcaagttatctatcttccaaaaggaaaaaccattcaacttcaagatttccgacgagtacggtgtcgatgaagccgctcacgcttgcgacgtcaagagatacggtcgtcactacctaagattggaccatgctattcacggtgttggctccgaagcctgtggtccagctgttttggaccaatacagactaaaggctcaagacttcaacttcgaatttgacttggctttcgaatag

SEQ ID NO: 19 - DNA, 60 bp, artificial sequence
  H98 primer
ttaccctaccagcaatataagtaaaaaactatgtcttgtttgatcccagaaaacctaaga

SEQ ID NO: 20 - DNA, 17 bp, artificial sequence
  M13ForTOPO primer
gtaaaacgacggccagt

SEQ ID NO: 21 - DNA, 22 bp, artificial sequence
  M13RevTOPO primer
cacacaggaaacagctatgacc

SEQ ID NO: 22 - DNA, 60 bp, artificial sequence
  H99 primer
agtttccagcacttgatattattttcctctctattcgaaagccaagtcaaattcgaagtt

SEQ ID NO: 23 - DNA, 9585 bp, artificial sequence
  PmeI digested pHR81-ILV5p-R8B2y2 plasmid
ggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcgaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttaaacagtatgaaggttttctacgacaaggattgtgacttgtctatcattcaaggtaaaaaggtcgccatcatcggttttggttcccaaggtcacgctcaagccttgaacttaaaggactctggtgtcgatgttaccgtcggtctaccaaagggtttcgctgacgttgccaaggccgaagctcacggtttcaaggttactgacgtcgccgctgccgttgctggtgctgatttggtcatgatcctaattccagacgaattccaatcccaattgtacaaaaacgaaatcgaaccaaacatcaaaaagggtgccactttggctttctcccacggtttcgctatccactacaaccaagttgttccaagagctgacttggacgttatcatgattgctcctaaggctccaggtcataccgttagatctgaattcgtcaagggtggtggtatcccagacttgattgctgtttaccaagacgtttctggtaatgccaaaaacgtcgctttgtcctacgctgccggtgttggtggtggtcgtactggtatcatcgaaactaccttcaaggacgaaaccgaaaccgacttattcggtgaacaagctgttttgtgtggtggtaccgtcgaattggtcaaggctggttttgaaactttggtcgaagctggttacgctccagaaatggcttacttcgaatgtttacacgaattgaagttgattgttgatttgatgtacgaaggtggtattgctaacatgaactactctatctctaacaacgctgaatacggtgaatacgttactggtccagaagtcattaacgccgaatctagacaagctatgagaaatgctttgaagagaattcaagatggtgaattcgctaagatgttcatctctgaaggtgctaccggttacccttctatgactgctaagcgtagaaacaacgctgctcacggtatcgaaatcatcggtgaacaactaagagctatgatgccatggattggtgctaacaagatcgtcgataagagaaaaaactgaaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatgtgtgcgtgtatgtgtacacctgtatttaatttccttactcgcgggtttttcttttttctcaattcttggcttcctctttctcgagcggaccggatcctccgcggtgccggcagatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagacaacatcatccacggttctatactgttgacccaatgcatctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatctcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactag

SEQ ID NO: 24 - DNA, 1089 bp, Saccharomyces cerevisiae
tttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcgaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaact

SEQ ID NO: 25 - DNA, 627 bp, Saccharomyces cerevisiae
agaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatgtgtgcgtgtatgtgtacacctgtatttaatttccttactcgcgggtttttcttttttctcaattcttggcttcctcttt

SEQ ID NO: 26 - DNA, 1122 bp, Escherichia coli
atgtctaaggttgctttgatcactggtgttacaggtcaagacggttcttacttggctgagttcttgttagaaaagggttacgaagtacacggcatcaagagacgtgcttctagcttcaacaccgaaagagttgaccatatctaccaagacccacacacttgtaacccaaagttccacttgcattacggtgatttgtccgatacttccaacttgaccagaatcttgagagaagtccaaccagatgaagtctacaacttgggtgctatgtctcacgtcgctgtttccttcgaatctcctgaatacaccgctgacgttgacgctatgggtaccttaagattgctagaagctatcagattcctaggcttggaaaagaaaaccaggttttaccaagcctccacctctgaattgtacggtttggtgcaagaaattccacaaaaggaaaccactccattctacccaagatccccatacgccgtcgctaagttgtacgcctactggatcaccgtcaattacagagaatcttacggtatgtacgcttgtaacggtatcttattcaaccacgaatccccaagacgtggtgaaaccttcgtcactagaaagatcaccagagccattgctaacattgctcaaggtttggaatcctgtttgtacttgggtaacatggactctttgagagactggggtcacgccaaggactacgttaagatgcaatggatgatgttgcaacaagaacaaccagaagatttcgttattgctacaggtgttcaatactctgtcagacaattcgtcgaaatggctgccgctcaattgggtatcaagttaagattcgaaggtactggtgtcgaagagaagggtattgttgtctcagttactggtcacgacgctccaggtgtcaagcctggtgacgtcatcattgctgttgatccaagatacttcagacccgctgaagttgaaactttgctaggtgacccaaccaaggcccacgaaaagttgggttggaagccagaaatcactttaagagaaatggtttctgaaatggtcgctaacgatttggaagctgccaagaaacactctttgctaaagtctcacggttacgacgttgccatcgctttggaaagttag

SEQ ID NO: 27 - DNA, 966 bp, Escherichia coli
atgtctaagcaaagagttttcatcgctggtcacagaggtatggtcggtagcgctatcaagagacaattggaacaaagaggcgacgtcgaattggtcttgagaaccagagatgaattgaacttgttagactcaagagctgttcacgacttctttgcctctgaatctatcgaccaagtttacctagctgccgctaaggtcggtggcatcgttgctaacaatacctacccagctgatttcatttaccaaaacatgatgatcgaatctaacatcattcacgctgcccaccaaaatgacgtcaacaaattgctattcttaggtagttcttgtatctaccctaagttggccaagcaaccaatggctgaatccgaattgctacaaggtactttggaaccaaccaacgaaccatacgctattgctaagatcgctggtatcaagttgtgtgaatcctacaacagacaatacggtagagactacagatccgtcatgccaaccaacttgtacggtccccacgacaacttccacccatccaactctcacgttatccctgccttgttaagacgtttccacgaagctaccgctcaaaacgctccagacgttgtcgtgtggggttctggtaccccaatgagagagttcttgcacgttgacgatatggctgccgcttctattcacgttatggaattagctcacgaagtttggttggaaaacactcaaccaatgttgtcccatatcaacgttggtactggtgtcgattgtactattagagaattggctcaaacaattgccaaggttgtcgattacaagggtagagttgtcttcgacgcttctaagccagacggtactccaagaaagttgctagacgtcaccagattgcaccaattgggttggtaccatgaaatctccttagaagctggtttggcctctacttaccaatggttcttggaaaaccgtgatcgtttcagaggttag

SEQ ID NO: 28 - DNA, 60 bp, artificial sequence
  H17 primer
acgcaaaataacacagtcaaatcaatcaaaatgtctaaggttgctttgatcactggtgtt

SEQ ID NO: 29 - DNA, 60 bp, artificial sequence
  H18 primer
tcataaatcataagaaattcgcttactcctaactttccaaagcgatggcaacgtcgtaac

SEQ ID NO: 30 - DNA, 60 bp, artificial sequence
  H15 primer
ataaccataaccaagtaatacatattcaaatgtctaagcaaagagttttcatcgctggtc

SEQ ID NO: 31 - DNA, 60 bp, artificial sequence
  H16 primer
aagatttaaagtaaattcacggccctgcaggctaacctctgaaacgatcacggttttcca

SEQ ID NO: 32 - DNA, 1713 bp, Saccharomyces cerevisiae
acctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcccgcacgccgaaatgcatgcaagtaacctattcaaagtaatatctcatacatgtttcatgagggtaacaacatgcgactgggtgagcatatgttccgctgatgtgatgtgcaagataaacaagcaaggcagaaactaacttcttcttcatgtaataaacacaccccgcgtttatttacctatctctaaacttcaacaccttatatcataactaatatttcttgagataagcacactgcacccataccttccttaaaaacgtagcttccagtttttggtggttccggcttccttcccgattccgcccgctaaacgcatatttttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatcaatcaaa

SEQ ID NO: 33 - DNA, 316 bp, Saccharomyces cerevisiae
gagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggt

SEQ ID NO: 34 - DNA, 725 bp, artificial sequence
  PGK(UAS)-FBA1 hybrid promoter
aattaccgtcgctcgtgatttgtttgcaaaaagaacaaaactgaaaaaacccagacacgctcgacttcctgtcttcctattgattgcagcttccaatttcgtcacacaacaaggtcctgtcgacgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaa

SEQ ID NO: 35 - DNA, 580 bp, Saccharomyces cerevisiae
gtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggataacgattctgaagttactcttagcgttcctatcggtacagccatcaaatcatgcctataaatcatgcctatatttgcgtgcagtcagtatcatctacatgaaaaaaactcccgcaatttcttatagaatacgttgaaaattaaatgtacgcgccaagataagataacatatatctagatgcagtaatatacacagattcccgcggacgtgggaaggaaaaaattagataacaaaatctgagtgatatggaaattccgctgtatagctcatatctttccct

SEQ ID NO: 36 - DNA, 1122 bp, Arabidopsis thaliana
atggcttctgaaaacaatggttctagatccgactctgaatctatcaccgctccaaaggctgattccaccgttgtcgaaccaagaaagatcgctttgatcactggtattactggtcaagatggttcatacttgactgagttcttgttaggcaagggttacgaagttcacggtttgatcagacgttctagcaacttcaacacccaaagaatcaatcacatctacattgatccacacaacgttaacaaggctttgatgaagttgcactacgccgacttaaccgacgcttcttccttgagacgttggattgatgttatcaaaccagacgaagtttacaacttggctgcccaatctcacgttgctgtttctttcgaaattccagactacactgctgacgttgtcgctacaggcgctttgagattgctagaagccgtcagatcccacactattgattctggtagaactgtcaagtactaccaagccggttcttccgaaatgttcggttctaccccacctccccaatctgaaaccactcctttccacccaagatccccatacgctgcctctaagtgtgctgcccactggtacactgtcaactacagagaagcctacggtttgtttgcttgtaacggtatcttgttcaaccatgaatccccaagacgtggtgaaaacttcgttaccagaaagataaccagagctttaggtagaatcaaggttggtttgcaaactaagttattcttgggtaacttgcaagctagtagagactggggtttcgctggtgactacgtggaagctatgtggttgatgttgcaacaagaaaagccagacgattacgttgtagctaccgaagagggtcacaccgttgaagagttcctagacgtctctttcggttacttgggtttgaactggaaggactacgtcgaaatcgaccaaagatactttagaccagctgaagttgacaacttacaaggtgacgcttccaaagccaaggaagtcttgggttggaagccacaagtcggtttcgaaaagttggttaagatgatggtcgatgaagacttggaactagctaagagagaaaaggtcctagtcgatgctggttacatggacgccaagcaacaaccatag

SEQ ID NO: 37 - DNA, 972 bp, Arabidopsis thaliana
atggctgaaaccatcggttctgaagttagctctatgtccgacaagtccgccaagatattcgtcgctggtcacagaggtttggtcggttccgctattgtcagaaagttgcaagaacaaggtttcaccaacctagttttgaagacccacgctgaactagacttgactagacaagctgacgttgaatctttcttttcccaagaaaagccagtctacgtcatcttggctgccgctaaggtcggtggcatccacgccaacaatacctacccagctgatttcattggtgttaacttgcaaatccaaacaaacgttattcactccgcttacgaacacggtgtcaagaaattgctattcttgggttcttcctgtatctacccaaagttcgctccacaacctatcccagaatctgctttgttaaccgcttctttggaacctaccaacgaatggtacgctatcgctaagattgccggtatcaagacttgtcaagcctacagaatccaacacggttgggatgctatctctggtatgccaaccaatttgtacggtccaaacgacaacttccacccagaaaacagtcacgttttgccagccctaatgagacgtttccatgaagccaaggtcaacggtgctgaagaggttgtcgtgtggggtactggttctcccttgagagagttcttgcacgttgacgatttggctgacgcctgtgtcttcttgttagatagatactcaggtttggaacacgtcaacatcggctctggtcaagaagttactattagagaattagctgaattggttaaggaagttgtcggtttcgaaggtaagttgggttgggactgtaccaagccagatggtactccacgtaagttgatggactcttccaaattggcttccttaggttggactccaaaggtttctttgagagacggtttgtctcaaacttacgactggtacttaaagaacgtttgcaacagatag

SEQ ID NO: 38 - DNA, 60 bp, artificial sequence
  H11 primer
ataaccataaccaagtaatacatattcaaatggctgaaaccatcggttctgaagttagct

SEQ ID NO: 39 - DNA, 60 bp, artificial sequence
  H12 primer
atgcaagatttaaagtaaattcacggccctgcaggctatctgttgcaaacgttctttaag

SEQ ID NO: 40 - DNA, 60 bp, artificial sequence
  H13 primer
cgcaaaataacacagtcaaatcaatcaaaatggcttctgaaaacaatggttctagatccg

SEQ ID NO: 41 - DNA, 60 bp, artificial sequence
  H14 primer
atcataaatcataagaaattcgcttactcctatggttgttgcttggcgtccatgtaacca

SEQ ID NO: 42 - DNA, 920 bp, artificial sequence
  coding sequence for a FutC enzyme from Helicobacter pylori with BsaI sites on ends
ggtctcaaggtatggctttcaaggttgttcaaatctgtggtggtttgggcaaccaaatgttccaatacgctttcgccaaatctttgcaaaagcacctaaacaccccagttttgttggatatcacctctttcgactggtccaacagaaagatgcaattggaattgttcccaatcgacttaccatacgcttctgccaaggaaattgctatcgccaaaatgcaacacttaccaaagttggtcagagataccttaaagtgtatgggtttcgacagagtttcccaagaaattgtcttcgagtacgaaccaggtttgctaaagccttccagattgacctacttttacggttacttccaagacccaagatacttcgatgctatttccccattgatcaagcaaactttcaccttgccacctcccgaaaatggcaacaacaagaagaaagaagaagaataccacagaaagttggctctaatcttagccgctaagaactctgttttcgtccacgtcagacgtggcgactacgttggtattggctgtcaattgggtatcgactaccaaaagaaggctttggaatacattgccaagagagtcccaaacatggaactatttgttttctgcgaagacttgaagttcactcaaaacttggacttgggttacccattcatggatatgactacaagagacaaggaagaagaggcttactgggatatgttgttaatgcaatcttgcaagcacggtatcatcgccaactccacttactcttggtgggccgcttacttgatcaacaatccagaaaagatcatcattggtccaaagcattggttgttcggtcacgaaaacattttgtgcaaagaatgggtcaagatcgagtctcacttcgaagtcaagtctaagaagtacaacgcctagtctagagagacc

SEQ ID NO: 43 - DNA, 45 bp, artificial sequence
  H305 primer
aattcggtcgaaaaaagaaaaggaggcagattgtactgagagtgc

SEQ ID NO: 44 - DNA, 45 bp, artificial sequence
  H306 primer
caggcaagtgcacaaacaatacttagtgcggtatttcacaccgca

SEQ ID NO: 45 - DNA, 20 bp, artificial sequence
  H291 primer
gccagaggtcaagccagaag

SEQ ID NO: 46 - DNA, 20 bp, artificial sequence
  H292 primer
ccgttgtcgtcgttcttcaa